(19) United States
(12) Patent Application Publication
(10) Pub. No.: US 2011/0262462 A1
(43) Pub. Date: Oct. 27, 2011

(54) POLYMORPHISM MARKERS FOR PREDICTING RESPONSE TO INTERLEUKIN-6 RECEPTOR-INHIBITING MONOCLONAL ANTIBODY DRUG TREATMENT

(75) Inventors: Adam Platt, Ely (GB); Laurent Essioux, Attenschwiller (FR); Mitchell Martin, Verona, NJ (US); Soren Germer, Maplewood, NJ (US); Jianmei Wang, Welwyn Garden City (GB)

(73) Assignee: Roche Molecular Systems, Inc., Pleasanton, CA (US)

(21) Appl. No.: 13/088,236

(22) Filed: Apr. 15, 2011

Related U.S. Application Data
(60) Provisional application No. 61/325,120, filed on Apr. 16, 2010.

(51) Int. Cl. A61K 39/395; C12Q 1/68; A61P 29/00; C40B 40/06

(52) U.S. Cl. 424/173.1; 506/16; 435/6.11

(57) ABSTRACT

The present invention provides single nucleotide polymorphisms (SNPs) associated with clinical responsiveness of rheumatoid arthritis patients to treatment with an interleukin-6 receptor antibody such as tocilizumab, and methods of using such SNPs for predicting clinical response to treatment with the antibody.

### CROSS-REFERENCE TO RELATED APPLICATIONS

This application claims benefit of U.S. provisional application No. 61/325,120, filed Apr. 16, 2010, which application is herein incorporated by reference in its entirety for all purposes.

### BACKGROUND OF THE INVENTION

Tocilizumab is the first humanized interleukin-6 receptor (IL-6R)-inhibiting monoclonal antibody that has been developed to treat rheumatoid arthritis. As with other treatments, the antibody exhibits a range of therapeutic efficacy in patients. Thus, there is a need to determine those patients that are more likely to respond positively to treatment with tocilizumab, and/or to identify those patients who are unlikely to respond to treatment with tocilizumab. The present invention addresses this need.

### BRIEF SUMMARY OF THE INVENTION

The invention is based, in part, on the discovery of single nucleotide polymorphisms (SNPs) that are associated with a positive therapeutic response to treatment with a humanized IL-6R-inhibiting monoclonal antibody such as tocilizumab. The invention therefore relates to the identification of SNPs, as well as combinations of such SNPs and haplotypes of SNPs, that are associated with predicting a rheumatoid arthritis patient's response to treatment with an IL-6R-inhibiting monoclonal antibody such as tocilizumab. Thus, in one aspect, the invention provides a method of identifying a patient that has an increased likelihood of responding to an IL-6R antibody, e.g. tocilizumab, the method comprising determining the presence of at least one SNP allele, or a polymorphism in strong linkage disequilibrium with the SNP, that is associated with a positive clinical outcome, wherein the SNP is selected from the SNPs listed in Table 1. In a further aspect, the invention provides a method of identifying a patient that should be excluded from treatment, the method comprising determining the presence of at least one SNP allele, or a polymorphism in strong linkage disequilibrium with the SNP, that is associated with a negative clinical out come, wherein the SNP is selected from the SNPs listed in Table 1.

In another aspect, the invention additionally provides devices and/or kits for determining the presence of one or more SNP alleles associated with positive and/or negative therapeutic response to treatment with an IL-6R-inhibiting monoclonal antibody such as tocilizumab. Such a device can, e.g., include an array (or microarray) that comprises nucleic acid probes selective for SNP alleles set forth in Table 1.

In another aspect, the invention provides methods of generating a report on the likelihood of a rheumatoid arthritis patient exhibiting a positive therapeutic response to an IL-6R-inhibiting monoclonal antibody such as tocilizumab.

In a further aspect of the invention, the SNPs and haplotypes described herein may also be used to predict the likelihood of a positive or negative therapeutic outcome to treatment with any therapeutic molecule that disrupts IL-6 signaling.

### DETAILED DESCRIPTION OF THE INVENTION

A “single nucleotide polymorphism (SNP) biomarker” refers to a SNP, or a polymorphism that is in strong linkage disequilibrium with the SNP, where an allele of the polymorphism is associated with the presence of a therapeutic response, often a positive therapeutic response, to a therapeutic treatment.

A “synonymous codon change” or a polymorphism that is “synonymous” refers to a change in a nucleic acid sequence that does not result in an alteration in protein sequence.

Terms such as “SNP,” “polymorphism,” “mutation, or “variation” are used interchangeably unless it is expressly indicated otherwise. Thus, an “allelic variant” in the context of this invention is an alternative form of a polymorphic allele.

As used herein, references to SNPs and SNP genotypes include individual SNPs and/or haplotypes, which are groups of SNPs that are generally inherited together.

A “SNP profile” of a rheumatoid arthritis patient, as used herein, refers to the pattern of alleles that a rheumatoid arthritis patient possesses at one or more of the SNPs set forth in Table 1, or a polymorphism that is in strong linkage disequilibrium (LD) with a SNP set forth in Table 1.

The term “a polymorphism in strong linkage disequilibrium with a SNP” refers to a polymorphism that is in linkage disequilibrium with a SNP identified herein, e.g., a SNP set forth in Table 1, where the linkage disequilibrium value is such that r²≧0.8. Linkage disequilibrium values can be calculated using various methods (see, e.g., Devlin & Risch, Genomics 29:311-322, 1995; and Jorde, Genome Res. 10:1435-1444, 2000 for reviews; see also Lewontin, Genetics 120:849-52, 1988; Hill & Robertson, Genet Res. 8:269-94, 1966). Data for determining LD can be obtained from various sources, e.g., from the HapMap project (The International HapMap Consortium. A second generation human haplotype map of over 3.1 million SNPs. Nature 449:851-61, 2007; see also Barrett, et al., Haploview: analysis and visualization of LD and haplotype maps. Bioinformatics 21:263-5, 2005.)

As used herein, a “positive therapeutic response” or “therapeutic benefit” refers to an improvement in, and/or delay in the onset of, any symptom of rheumatoid arthritis.

As used herein “negative therapeutic response” refers to a lack of improvement of one or more symptoms of rheumatoid arthritis; or in some embodiments, refers to an adverse side effect such as increases in LDL or a serious infection adverse event.

An “interleukin-6 receptor (IL-6R) inhibiting antibody” refers to an antibody to IL-6 receptor where the antibody binds to IL-6 receptor and antagonizes (i.e., inhibits) IL-6 receptor activity. An example of such an antibody is tocilizumab, a humanized IL-6R monoclonal antibody (see, e.g., Sato et al., Cancer Res 1993; 53: 851-6; and U.S. Pat. No. 7,479,543) that is used for the treatment of rheumatoid arthritis.

The term “hybridization” refers to the formation of a duplex structure by two single stranded nucleic acids due to complementary base pairing. Hybridization can occur between exactly complementary nucleic acid strands or between nucleic acid strands that contain minor regions of mismatch. As used herein, the term “substantially complementary” refers to sequences that are complementary except for minor regions of mismatch. Typically, the total number of mismatched nucleotides over a hybridizing region is not more than 3 nucleotides for sequences about 15 nucleotides in length. Conditions under which only exactly complementary nucleic acid strands will hybridize are referred to as “stringent” or “sequence-specific” hybridization conditions. Stable duplexes of substantially complementary nucleic acids can be achieved under less stringent hybridization conditions. Those skilled in the art of nucleic acid technology can determine duplex stability empirically considering a number of variables including, for example, the length and base pair concentration of the oligonucleotides, ionic strength, and incidence of mismatched base pairs. For example, computer software for calculating duplex stability is commercially available from National Biosciences, Inc. (Plymouth, Minn.); e.g., OLIGO version 5, or from DNA Software (Ann Arbor, Mich.), e.g., Visual OMP 6.

Stringent, sequence-specific hybridization conditions, under which an oligonucleotide will hybridize only to the exactly complementary target sequence, are well known in the art (see, e.g., the general references provided in the section on detecting polymorphisms in nucleic acid sequences). Stringent conditions are sequence-dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the base pairs have dissociated. Relaxing the stringency of the hybridizing conditions will allow sequence mismatches to be tolerated; the degree of mismatch tolerated can be controlled by suitable adjustment of the hybridization conditions.

The term “primer” refers to an oligonucleotide that acts as a point of initiation of DNA synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature. A primer is preferably a single-stranded oligodeoxyribonucleotide. The primer includes a “hybridizing region” exactly or substantially complementary to the target sequence, preferably about 15 to about 35 nucleotides in length. A primer oligonucleotide can either consist entirely of the hybridizing region or can contain additional features which allow for the detection, immobilization, or manipulation of the amplified product, but which do not alter the ability of the primer to serve as a starting reagent for DNA synthesis. For example, a nucleic acid sequence tail can be included at the 5′ end of the primer that hybridizes to a capture oligonucleotide.

An “allele-specific” primer, as used herein, is a primer that hybridizes to a target sequence such that the 3′ end, usually the 3′ nucleotide, of the primer aligns with a site of interest, e.g., a SNP site, and is exactly complementary to either the major allele or a minor allele at the position of interest. As used herein, the primer is “specific for” the allele to which it is exactly complementary at the 3′ end, e.g., at the 3′ nucleotide or penultimate nucleotide. In general, primer extension is inhibited when a mismatch is present at the 3′ end of the primer. An allele-specific primer, when hybridized to the exactly complementary allele, is extendable at a greater efficiency. The same primer, when hybridized to the other allele, is not as readily extendable because of the mismatch at the 3′ end, e.g., the 3′ nucleotide or penultimate nucleotide at the 3′ end, of the primer in the hybridization duplex. Thus, the use of an allele-specific primer provides allelic discrimination based on differential formation of an extension product.

The term “probe” refers to an oligonucleotide that selectively hybridizes to a target nucleic acid under suitable conditions.

An “allele-specific” probe contains a “hybridizing region” exactly or substantially complementary to the target sequence, and is exactly complementary to the target sequence at the site of interest, e.g., a SNP polymorphic site. Thus, for example, an allele-specific probe for one SNP allele selectively detects one of the nucleotides at the polymorphic site, whereas a different allele-specific probe selectively detects an alternative nucleotide present at the polymorphic site. A hybridization assay carried out using the probe under sufficiently stringent hybridization conditions enables the selective detection of a specific target sequence comprising the site of interest. The probe hybridizing region is preferably from about 10 to about 35 nucleotides in length, more preferably from about 15 to about 35 nucleotides in length. The use of modified bases or base analogues which affect the hybridization stability, which are well known in the art, may enable the use of shorter or longer probes with comparable stability. A probe oligonucleotide can either consist entirely of the hybridizing region or can contain additional features which allow for the detection or immobilization of the probe, but which do not significantly alter the hybridization characteristics of the hybridizing region.

The term “target sequence” or “target region” refers to a region of a nucleic acid that is to be analyzed and comprises the polymorphic site of interest.

As used herein, the terms “nucleic acid,” “polynucleotide” and “oligonucleotide” refer to primers, probes, and oligomer fragments. The terms are not limited by length and are generic to linear polymers of polydeoxyribonucleotides (containing 2-deoxy-D-ribose), polyribonucleotides (containing D-ribose), and any other N-glycoside of a purine or pyrimidine base, or modified purine or pyrimidine bases. These terms include double- and single-stranded DNA, as well as double- and single-stranded RNA. Oligonucleotides for use in the invention may be used as primers and/or probes.

A nucleic acid, polynucleotide or oligonucleotide can comprise phosphodiester linkages or modified linkages including, but not limited to phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

A nucleic acid, polynucleotide or oligonucleotide can comprise the five biologically occurring bases (adenine, guanine, thymine, cytosine and uracil) and/or bases other than the five biologically occurring bases. These bases may serve a number of purposes, e.g., to stabilize or destabilize hybridization; to promote or inhibit probe degradation; or as attachment points for detectable moieties or quencher moieties. For example, a polynucleotide of the invention can contain one or more modified, non-standard, or derivatized base moieties, including, but not limited to, N6-methyl-adenine, N6-tert-butyl-benzyl-adenine, imidazole, substituted imidazoles, 5-fluorouracil, 5 bromouracil, 5-chlorouracil, 5-iodouracil, hypoxanthine, xanthine, 4-acetylcytosine, 5 (carboxyhydroxymethyl)uracil, 5 carboxymethylaminomethyl-2-thiouridine, 5 carboxymethylaminomethyluracil, dihydrouracil, beta-D-galactosylqueosine, inosine, N6 isopentenyladenine, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2 thiocytosine, 5-methyl-2-thiouracil, 2-thiouracil, 4-thiouracil, 5-methyluracil, uracil-5-oxyacetic acidmethylester, 3-(3-amino-3-N-2-carboxypropyl)uracil, (acp3)w, 2,6-diaminopurine, and 5-propynyl pyrimidine. Other examples of modified, non-standard, or derivatized base moieties may be found in U.S. Pat. Nos. 6,001,611; 5,955,589; 5,844,106; 5,789,562; 5,750,343; 5,728,525; and 5,679,785, each of which is incorporated herein by reference in its entirety. Furthermore, a nucleic acid, polynucleotide or oligonucleotide can comprise one or more modified sugar moieties including, but not limited to, arabinose, 2-fluoroarabinose, xylulose, and a hexose.

The terms “arrays,” “microarrays,” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. The arrays are prepared using known methods.

### INTRODUCTION

The invention is based, at least in part, on the identification of a number of SNPs (see, e.g., Table 1) that are associated with magnitude of clinical response of rheumatoid arthritis patients to treatment with an IL-6R antibody, tocilizumab. Thus, the invention provides methods and devices for analyzing for the presence of particular SNP alleles to use in the determination of rheumatoid arthritis patients that are likely to have a beneficial response to treatment with an IL-6R antibody, preferably tocilizumab; or to identify those patients that have a SNP pattern that is associated with a poor clinical response to treatment with an IL-6R antibody such as tocilizumab. Further, detection of at least one SNP set forth in Table 1 may also be used to predict endpoints other than rheumatoid arthritis symptoms, e.g., changes in Low Density Lipoprotein (ΔLDL) or a Serious Infection Adverse Event (SIAE) following dosing with IL-6R antibody, e.g., tocilizumab.

The identification of a gene/transcript/protein/metabolite, linked by pathway or cell type or tissue expression to the SNPs identified in this analysis, may also be used as an alternative biomarker for measurement of response to treatment with an IL-6R antibody such as tocilizumab. Similarly, the identification of a gene/transcript/protein/metabolite, linked by pathway or cell type or tissue expression to one or more SNPs identified in Table 1 may also be used as an alternative biomarker to predict ΔLDL or a SIAE following dosing with tocilizumab, or other IL-6R antibody therapeutic agent.

The contribution or association of particular SNPs and/or SNP haplotypes with positive responses to treatment with tocilizumab provides the basis of diagnostic/prognostic tests to identify patients who are more likely to respond to therapy with an IL-6 receptor antibody. In the current invention, the diagnostic/prognostic analysis may be based on a single SNP or a group of SNPs. Combined detection of a plurality of SNPs (for example, 2, 3, 4, 5, 6, 7, 8, 9, 10, 15, 20, 25, 30, 35, 50, 100, 150, 200, 250, or 300, or 319, or any other number in-between, or more, of the SNPs provided in Table 1) is typically preferable for increased predictive power. SNP analysis may also be combined with other prognostic indicators, e.g., pathological characteristics, to assist in identifying patients that are likely to respond to treatment with tocilizumab, or another IL-6 receptor antibody used for the treatment of rheumatoid arthritis; or another therapy for rheumatoid arthritis that disrupts IL-6 signaling.

Similarly, SNP analysis may also serve as a prognostic indicator for those patients who would not likely benefit from treatment with an IL-6R antibody such as tocilizumab.

The process of determining which nucleotide(s) is/are present at each of one or more SNP positions (such as a SNP position disclosed in Table 1), for either or both alleles, may be referred to by such phrases as SNP genotyping, determining the “identity” of a SNP, determining the “content” of a SNP, or determining which nucleotide(s)/allele(s) is/are present at a SNP position. Thus, these terms can refer to detecting a single allele (nucleotide) at a SNP position or can encompass detecting both alleles (nucleotides) at a SNP position (such as to determine the homozygous or heterozygous state of a SNP position). Furthermore, in some embodiments, these terms may also refer to detecting an amino acid residue encoded by a SNP (such as alternative amino acid residues that are encoded by different codons created by alternative nucleotides at a SNP position).

The SNPs shown in Table 1 include SNPs that are associated with a positive therapeutic outcome as well as those associated with a negative therapeutic outcome. Thus, in some embodiments, patients are excluded from treatment to IL-6R antibody, e.g., tocilizumab, based on the SNP alleles present in the patient.

In some embodiments, the SNPs that are analyzed are one or more of the seven SNPs set forth in Table 2. In some embodiments, two of the seven SNPs are analyzed. In additional embodiments, three, four, five, six, or all of the seven SNPs are analyzed.

In Table 1, the beta value reflects the association (negative or positive) with the number of minor SNP alleles. Thus, for example, if the presence of a minor allele (in a patient being tested) of a SNP in Table I is detected, this is indicative of a positive response to the IL-6R antagonist, e.g., tocilizumab if the SNP is shown as having a negative beta for a symptom of RA.

### Detection of Nucleic Acid Sequence Polymorphisms

Detection techniques for evaluating nucleic acids for the presence of a SNP involve procedures well known in the field of molecular genetics. Many of the methods involve amplification of nucleic acids. Ample guidance for performing methods of SNP analysis is readily available in the art. Exemplary references include manuals such as PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Current Protocols in Molecular Biology, Ausubel, 1994-1999, including supplemental updates through April 2010; Sambrook & Russell, Molecular Cloning, A Laboratory Manual (3rd Ed, 2001).

Although SNP allele detection methods typically employ PCR steps, other amplification protocols may also be used. Suitable amplification methods include ligase chain reaction (see, e.g., Wu & Wallace, Genomics 4:560-569, 1988); strand displacement assay (see, e.g., Walker et al., Proc. Natl. Acad. Sci. USA 89:392-396, 1992; U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., Proc. Natl. Acad. Sci. USA 86:1173-1177, 1989); and self-sustained sequence replication (3SR) (Guatelli et al., Proc. Natl. Acad. Sci. USA 87:1874-1878, 1990; WO 92/08800). Alternatively, methods that amplify a probe to detectable levels can be used, such as Qβ-replicase amplification (Kramer & Lizardi, Nature 339:401-402, 1989; Lomeli et al., Clin. Chem. 35:1826-1831, 1989). A review of known amplification methods is provided, for example, by Abramson and Myers in Current Opinion in Biotechnology 4:41-47, 1993.

In numerous embodiments, the detection of the genotype at a SNP biomarker, e.g., of a SNP set forth in Table 1, of an individual is performed using oligonucleotide primers and/or probes. Oligonucleotides can be prepared by any suitable method, usually chemical synthesis, and can also be purchased through commercial sources. Oligonucleotides can include modified phosphodiester linkages (e.g., phosphorothioate, methylphosphonates, phosphoamidate, or boranophosphate) or linkages other than a phosphorous acid derivative oligonucleotide may be used to prevent cleavage at a selected site. In addition, the use of 2′-amino modified sugars tends to favor displacement over digestion of the oligonucleotide when hybridized to a nucleic acid that is also the template for synthesis of a new nucleic acid strand.

In some embodiments, one of skill in the art may determine expression levels of a transcript of a gene linked by pathway, cell-type, etc. to a SNP identified herein. The expression levels in a rheumatoid arthritis patient of a product encoded by a gene biomarker can be determined using many detection methods that are well known in the art.

Determination of expression level is often performed by analyzing a nucleic acid sample that is obtained from the individual to be analyzed. Often, the nucleic acid sample comprises messenger RNA.

In embodiments in which the SNP genotype is determined, the nucleic acid sample typically comprises genomic DNA, which can be obtained from peripheral blood lymphocytes or from other cells or tissues.

It is also possible to analyze RNA samples (and the cDNA transcribed therefrom) for the presence of polymorphic alleles where the polymorphism is present in a region that is transcribed. For example, mRNA can be used to determine the genotype of an individual at a transcribed polymorphic site. Such an analysis can be performed by first reverse-transcribing the target RNA using, for example, a viral reverse transcriptase, and then amplifying the resulting cDNA; or using a combined high-temperature reverse-transcription-polymerase chain reaction (RT-PCR), as described in U.S. Pat. Nos. 5,310,652; 5,322,770; 5,561,058; 5,641,864; and 5,693,517.

Frequently used methodologies for analysis of nucleic acid samples to detect SNPs are briefly described. However, any method known in the art can be used in the invention to detect the presence of single nucleotide polymorphisms.

### Allele Specific Hybridization

This technique, also commonly referred to as allele-specific oligonucleotide hybridization (ASO) (e.g., Stoneking et al., Am. J. Hum. Genet. 48:70-382, 1991; Saiki et al., Nature 324, 163-166, 1986; EP 235,726; and WO 89/11548), relies on distinguishing between two DNA molecules differing by one base by hybridizing an oligonucleotide probe that is specific for one of the variants to an amplified product obtained from amplifying the nucleic acid sample. This method typically employs short oligonucleotides, e.g., 15-20 bases in length. The probes are designed to differentially hybridize to one variant versus another. Principles and guidance for designing such probe is available in the art, e.g., in the references cited herein. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15-base oligonucleotide at the 7 position; in a 16-based oligonucleotide at either the 8 or 9 position) of the probe, but this design is not required.

The amount and/or presence of an allele is determined by measuring the amount of allele-specific oligonucleotide that is hybridized to the sample. Typically, the oligonucleotide is labeled with a label such as a fluorescent label. For example, an allele-specific oligonucleotide is applied to immobilized oligonucleotides representing one of the SNP allele sequences. After stringent hybridization and washing conditions, fluorescence intensity is measured for each SNP oligonucleotide.

In one embodiment, the nucleotide present at the polymorphic site is identified by hybridization under sequence-specific hybridization conditions with an oligonucleotide probe exactly complementary to one of the polymorphic alleles in a region encompassing the polymorphic site. The probe hybridizing sequence and sequence-specific hybridization conditions are selected such that a single mismatch at the polymorphic site destabilizes the hybridization duplex sufficiently so that it is effectively not formed. Thus, under sequence-specific hybridization conditions, stable duplexes will form only between the probe and the exactly complementary allelic sequence. Thus, oligonucleotides from about 10 to about 35 nucleotides in length, preferably from about 15 to about 35 nucleotides in length, which are exactly complementary to an allele sequence in a region which encompasses the polymorphic site are within the scope of the invention.

In an alternative embodiment, the nucleotide present at the polymorphic site is identified by hybridization under sufficiently stringent hybridization conditions with an oligonucleotide substantially complementary to one of the SNP alleles in a region encompassing the polymorphic site, and exactly complementary to the allele at the polymorphic site. Because mismatches which occur at non-polymorphic sites are mismatches with both allele sequences, the difference in the number of mismatches in a duplex formed with the target allele sequence and in a duplex formed with the corresponding non-target allele sequence is the same as when an oligonucleotide exactly complementary to the target allele sequence is used. In this embodiment, the hybridization conditions are relaxed sufficiently to allow the formation of stable duplexes with the target sequence, while maintaining sufficient stringency to preclude the formation of stable duplexes with non-target sequences. Under such sufficiently stringent hybridization conditions, stable duplexes will form only between the probe and the target allele. Thus, oligonucleotides from about 10 to about 35 nucleotides in length, preferably from about 15 to about 35 nucleotides in length, which are substantially complementary to an allele sequence in a region which encompasses the polymorphic site, and are exactly complementary to the allele sequence at the polymorphic site, are within the scope of the invention.

The use of substantially, rather than exactly, complementary oligonucleotides may be desirable in assay formats in which optimization of hybridization conditions is limited. For example, in a typical multi-target immobilized-probe assay format, probes for each target are immobilized on a single solid support. Hybridizations are carried out simultaneously by contacting the solid support with a solution containing target DNA. As all hybridizations are carried out under identical conditions, the hybridization conditions cannot be separately optimized for each probe. The incorporation of mismatches into a probe can be used to adjust duplex stability when the assay format precludes adjusting the hybridization conditions. The effect of a particular introduced mismatch on duplex stability is well known, and the duplex stability can be routinely both estimated and empirically determined, as described above. Suitable hybridization conditions, which depend on the exact size and sequence of the probe, can be selected empirically using the guidance provided herein and well known in the art. The use of oligonucleotide probes to detect single base pair differences in sequence is described in, for example, Conner et al., 1983, Proc. Natl. Acad. Sci. USA 80:278-282, and U.S. Pat. Nos. 5,468,613 and 5,604,099, each incorporated herein by reference.

The proportional change in stability between a perfectly matched and a single-base mismatched hybridization duplex depends on the length of the hybridized oligonucleotides. Duplexes formed with shorter probe sequences are destabilized proportionally more by the presence of a mismatch. In practice, oligonucleotides between about 15 and about 35 nucleotides in length are preferred for sequence-specific detection. Furthermore, because the ends of a hybridized oligonucleotide undergo continuous random dissociation and re-annealing due to thermal energy, a mismatch at either end destabilizes the hybridization duplex less than a mismatch occurring internally. Preferably, for discrimination of a single base pair change in target sequence, the probe sequence is selected which hybridizes to the target sequence such that the polymorphic site occurs in the interior region of the probe.

The above criteria for selecting a probe sequence that hybridizes to a particular SNP allele apply to the hybridizing region of the probe, i.e., that part of the probe which is involved in hybridization with the target sequence. A probe may be bound to an additional nucleic acid sequence, such as a poly-T tail used to immobilize the probe, without significantly altering the hybridization characteristics of the probe. One of skill in the art will recognize that for use in the present methods, a probe bound to an additional nucleic acid sequence which is not complementary to the target sequence and, thus, is not involved in the hybridization, is essentially equivalent to the unbound probe.

Suitable assay formats for detecting hybrids formed between probes and target nucleic acid sequences in a sample are known in the art and include the immobilized target (dot-blot) format and immobilized probe (reverse dot-blot or line-blot) assay formats. Dot blot and reverse dot blot assay formats are described in U.S. Pat. Nos. 5,310,893; 5,451,512; 5,468,613; and 5,604,099; each incorporated herein by reference.

In a dot-blot format, amplified target DNA is immobilized on a solid support, such as a nylon membrane. The membrane-target complex is incubated with labeled probe under suitable hybridization conditions, unhybridized probe is removed by washing under suitably stringent conditions, and the membrane is monitored for the presence of bound probe. In the reverse dot-blot (or line-blot) format, the probes are immobilized on a solid support, such as a nylon membrane or a microtiter plate. The target DNA is labeled, typically during amplification by the incorporation of labeled primers. One or both of the primers can be labeled. The membrane-probe complex is incubated with the labeled amplified target DNA under suitable hybridization conditions, unhybridized target DNA is removed by washing under suitably stringent conditions, and the membrane is monitored for the presence of bound target DNA.

An allele-specific probe that is specific for one of the polymorphism variants is often used in conjunction with the allele-specific probe for the other polymorphism variant. In some embodiments, the probes are immobilized on a solid support and the target sequence in an individual is analyzed using both probes simultaneously. Examples of nucleic acid arrays are described by WO 95/11995. The same array or a different array can be used for analysis of characterized polymorphisms. WO 95/11995 also describes subarrays that are optimized for detection of variant forms of a pre-characterized polymorphism. Such a subarray can be used in detecting the presence of a SNP described herein.

### Allele-Specific Primers

Polymorphisms are also commonly detected using allele-specific amplification or primer extension methods. These reactions typically involve use of primers that are designed to specifically target a polymorphism via a mismatch at the 3′ end of a primer. The presence of a mismatch effects the ability of a polymerase to extend a primer when the polymerase lacks error-correcting activity. For example, to detect an allele sequence using an allele-specific amplification- or extension-based method, a primer complementary to one of the specific alleles of a SNP is designed such that the 3′ terminal nucleotide hybridizes at the polymorphic position. The presence of the particular allele can be determined by the ability of the primer to initiate extension. If the 3′ terminus is mismatched, the extension is impeded. Thus, for example, if a primer matches the target allele nucleotide at the 3′ end, the primer will be efficiently extended.

Typically, the primer is used in conjunction with a second primer in an amplification reaction. The second primer hybridizes at a site unrelated to the polymorphic position. Amplification proceeds from the two primers leading to a detectable product signifying the particular allelic form is present. Allele-specific amplification- or extension-based methods are described in, for example, WO 93/22456; U.S. Pat. Nos. 5,137,806; 5,595,890; 5,639,611; and U.S. Pat. No. 4,851,331.

Using allele-specific amplification-based genotyping, identification of the alleles requires only detection of the presence or absence of amplified target sequences. Methods for the detection of amplified target sequences are well known in the art. For example, gel electrophoresis and probe hybridization assays described are often used to detect the presence of nucleic acids.

In an alternative probe-less method, the amplified nucleic acid is detected by monitoring the increase in the total amount of double-stranded DNA in the reaction mixture, is described, e.g., in U.S. Pat. No. 5,994,056; and European Patent Publication Nos. 487,218 and 512,334. The detection of double-stranded target DNA relies on the increased fluorescence various DNA-binding dyes, e.g., SYBR Green, exhibit when bound to double-stranded DNA.

As appreciated by one in the art, allele-specific amplification methods can be performed in reaction that employ multiple allele-specific primers to target particular alleles. Primers for such multiplex applications are generally labeled with distinguishable labels or are selected such that the amplification products produced from the alleles are distinguishable by size. Thus, for example, both alleles in a single sample can be identified using a single amplification by gel analysis of the amplification product.

As in the case of allele-specific probes, an allele-specific oligonucleotide primer may be exactly complementary to one of the polymorphic alleles in the hybridizing region or may have some mismatches at positions other than the 3′ terminus of the oligonucleotide, which mismatches occur at non-polymorphic sites in both allele sequences.

5′-nuclease Assay

Genotyping can also be performed using a “TaqMan®” or “5′-nuclease assay”, as described in U.S. Pat. Nos. 5,210,015; 5,487,972; and 5,804,375; and Holland et al., 1988, Proc. Natl. Acad. Sci. USA 88:7276-7280. In the TaqMan® assay, labeled detection probes that hybridize within the amplified region are added during the amplification reaction. The probes are modified so as to prevent the probes from acting as primers for DNA synthesis. The amplification is performed using a DNA polymerase having 5′ to 3′ exonuclease activity. During each synthesis step of the amplification, any probe which hybridizes to the target nucleic acid downstream from the primer being extended is degraded by the 5′ to 3′ exonuclease activity of the DNA polymerase. Thus, the synthesis of a new target strand also results in the degradation of a probe, and the accumulation of degradation product provides a measure of the synthesis of target sequences.

The hybridization probe can be an allele-specific probe that discriminates between the SNP alleles. Alternatively, the method can be performed using an allele-specific primer and a labeled probe that binds to amplified product.

Any method suitable for detecting degradation product can be used in a 5′ nuclease assay. Often, the detection probe is labeled with two fluorescent dyes, one of which is capable of quenching the fluorescence of the other dye. The dyes are attached to the probe, preferably one attached to the 5′ terminus and the other is attached to an internal site, such that quenching occurs when the probe is in an unhybridized state and such that cleavage of the probe by the 5′ to 3′ exonuclease activity of the DNA polymerase occurs in between the two dyes. Amplification results in cleavage of the probe between the dyes with a concomitant elimination of quenching and an increase in the fluorescence observable from the initially quenched dye. The accumulation of degradation product is monitored by measuring the increase in reaction fluorescence. U.S. Pat. Nos. 5,491,063 and 5,571,673, both incorporated herein by reference, describe alternative methods for detecting the degradation of probe which occurs concomitant with amplification.

### DNA Sequencing and Single Base Extensions

A SNP biomarker can also be detected by direct sequencing. Methods include e.g., dideoxy sequencing-based methods although other methods such as Maxam and Gilbert sequencing are also known (see, e.g., Sambrook and Russell, supra).

Sequencing detection methods include Pyrosequencing™ of oligonucleotide-length products. Such methods often employ amplification techniques such as PCR. For example, in pyrosequencing, a sequencing primer is hybridized to a single stranded, PCR-amplified, DNA template; and incubated with the enzymes, DNA polymerase, ATP sulfurylase, luciferase and apyrase, and the substrates, adenosine 5′ phosphosulfate (APS) and luciferin. The first of four deoxynucleotide triphosphates (dNTP) is added to the reaction. DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a Pyrogram™. Each light signal is proportional to the number of nucleotides incorporated. Apyrase, a nucleotide degrading enzyme, continuously degrades unincorporated dNTPs and excess ATP. When degradation is complete, another dNTP is added.

Another similar method for characterizing SNPs does not require use of a complete PCR, but typically uses only the extension of a primer by a single, fluorescence-labeled dideoxyribonucleic acid molecule (ddNTP) that is complementary to the nucleotide to be investigated. The nucleotide at the polymorphic site can be identified via detection of a primer that has been extended by one base and is fluorescently labeled (e.g., Kobayashi et al, Mol. Cell. Probes, 9:175-182, 1995).

The sequence can be determined using any other DNA sequencing method including, e.g., methods that use semiconductor technology to detect nucleotides that are incorporated into an extended primer by measuring changes in current that occur when a nucleotide is incorporated (see, e.g., U.S. Patent Application Publication Nos. 20090127589 and 20100035252). Other techniques include direct label-free exonuclease sequencing in which nucleotides cleaved from the nucleic acid are detected by passing through a nanopore (Oxford Nanopore) (Clark et al., Nature Nanotechnology 4: 265-270, 2009); and Single Molecule Real Time (SMRT™) DNA sequencing technology (Pacific Biosciences), which is a sequencing-by synthesis technique.

### Additional Techniques

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization—Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays.

Amplification products generated using the polymerase chain reaction can be analyzed by the use of denaturing gradient gel electrophoresis. Different alleles can be identified based on the different sequence-dependent melting properties and electrophoretic migration of DNA in solution (see, e.g., Erlich, ed., PCR Technology, Principles and Applications for DNA Amplification, W. H. Freeman and Co, New York, 1992, Chapter 7).

Alleles of target sequences can be differentiated using single-strand conformation polymorphism analysis, which identifies base differences by alteration in electrophoretic migration of single stranded PCR products, as described, e.g., in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target

SNP detection methods often employ labeled oligonucleotides. Oligonucleotides can be labeled by incorporating a label detectable by spectroscopic, photochemical, biochemical, immunochemical, or chemical means. Useful labels include fluorescent dyes, radioactive labels, e.g., ³²P, electron-dense reagents, enzyme, such as peroxidase or alkaline phosphatase, biotin, or haptens and proteins for which antisera or monoclonal antibodies are available. Labeling techniques are well known in the art (see, e.g., Current Protocols in Molecular Biology, supra; Sambrook & Russell, supra).

### Detection of Protein Variants

In some embodiments, e.g., where a biomarker allele results in a change of a protein sequence, it may be possible to detect variant alleles using methods that discriminate between the two variant proteins. Often these methods employ an antibody specific to the protein encoded by a variant allele.

A general overview of the applicable technology can be found in Harlow & Lane, Antibodies: A Laboratory Manual (1988) and Harlow & Lane, Using Antibodies (1999). Methods of producing polyclonal and monoclonal antibodies that react specifically with an allelic variant are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975)). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

Polymorphic alleles can be detected by a variety of immunoassay methods. For a review of immunological and immunoassay procedures, see Basic and Clinical Immunology (Stites & Terr eds., 7th ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Terr, eds., 7th ed. 1991).

Commonly used assays include noncompetitive assays, e.g., sandwich assays, and competitive assays. Typically, an assay such as an ELISA assay can be used. The amount of the polypeptide variant can be determined by performing quantitative analyses.

Other detection techniques, e.g., MALDI, may be used to directly detect the presence of a difference in protein sequence when comparing SNP alleles.

### Devices and Kits

In a further aspect, the invention provides diagnostic devices and kits for identifying SNPs associated with improved responsiveness of a rheumatoid arthritis patient to a therapeutic IL-6R antibody such as tocilizumab.

In some embodiments, a diagnostic device comprises probes to detect at least 2, 3, 4, 5, 6, 7, 8, 9, 10, 15, 20, or 50, or at least 100, at least 200, or at least 300 of the SNPs set forth in Table 1. In some embodiments, the present invention provides oligonucleotide probes attached to a solid support, such as a microarray slide or chip, e.g., as described in DNA Microarrays: A Molecular Cloning Manual, 2003, Eds. Bowtell and Sambrook, Cold Spring Harbor Laboratory Press. Construction of such devices are well known in the art, for example as described in Patents and Patent Publications U.S. Pat. No. 5,837,832; PCT application WO95/11995; U.S. Pat. No. 5,807,522; U.S. Pat. Nos. 7,157,229, 7,083,975, 6,444,175, 6,375,903, 6,315,958, 6,295,153, and 5,143,854, 2007/0037274, 2007/0140906, 2004/0126757, 2004/0110212, 2004/0110211, 2003/0143550, 2003/0003032, and 2002/0041420. Nucleic acid arrays are also reviewed in the following references: Biotechnol Annu Rev 8:85-101 (2002); Sosnowski et al, Psychiatr Genet. 12(4):181-92 (December 2002); Heller, Annu Rev Biomed Eng 4: 129-53 (2002); Kolchinsky et al, Hum. Mutat 19(4):343-60 (April 2002); and McGail et al, Adv Biochem Eng Biotechnol 77:21-42 (2002).

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. An array can be composed of a large number of unique, single-stranded polynucleotides. Typical polynucleotides are preferably about 6-60 nucleotides in length, more preferably about 15-30 nucleotides in length, and most preferably about 18-25 nucleotides in length. For certain types of arrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length. Polynucleotides used in the microarray or detection kit can be specific to a SNP or SNPs of interest (e.g., specific to a particular SNP allele at a target SNP site, or specific to particular SNP alleles at multiple different SNP sites), or specific to a polymorphic gene/transcript or genes/transcripts of interest.

A person skilled in the art will recognize that, based on the SNP and associated sequence information disclosed herein, detection reagents can be developed and used to assay any SNP set forth in Table 1, or a polymorphism in linkage disequilibrium (LD) with a SNP set forth in Table 1, individually or in combination, and that such detection reagents can be incorporated into a kit. The term “kit” as used herein in the context of SNP detection reagents, refers to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.). Accordingly, the present invention further provides SNP detection kits and systems, including but not limited to, packaged probe and primer sets (e.g., TaqMan probe/primer sets) for detecting one or more SNP alleles described herein, e.g., in Table 1 or Table 2, arrays/microarrays of nucleic acid molecules to detect one or more SNP alleles, and beads that contain one or more probes, primers, or other detection reagents for detecting one or more SNPs of the present invention. The kits can optionally include various electronic hardware components; for example, arrays (“DNA chips”) and microfluidic systems (“lab-on-a-chip” systems) provided by various manufacturers typically comprise hardware components. Other kits (e.g., probe/primer sets) may not include electronic hardware components, but may be comprised of, for example, one or more SNP detection reagents (along with, optionally, other biochemical reagents) packaged in one or more containers.

In some embodiments, a SNP detection kit typically contains one or more detection reagents and other components (e.g. a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a SNP-containing nucleic acid molecule. A kit may further contain means for determining the amount of a target nucleic acid, e.g., whether an individual is heterozygous or homozygous for a polymorphism or when detecting a gene transcript, and means for comparing the amount with a standard, and can comprise instructions for using the kit to detect the SNP-containing nucleic acid molecule of interest. In one embodiment of the present invention, kits are provided which contain the necessary reagents to carry out one or more assays to detect one or more SNPs disclosed herein. In a preferred embodiment of the present invention, SNP detection kits/systems are in the form of nucleic acid arrays, or compartmentalized kits, including microfluidic/lab-on-a-chip systems.

SNP detection kits/systems may contain, for example, one or more probes, or pairs of probes, that hybridize to a nucleic acid molecule at or near each target SNP position. Multiple pairs of allele-specific probes may be included in the kit/system to simultaneously assay multiple SNPs, preferably one or more SNPs set forth in Table 1. In some kits/systems, the allele-specific probes are immobilized to a substrate such as an array or bead. For example, the same substrate can comprise allele-specific probes for detecting at least 1, 10, 100, (or any other number in-between) or substantially all of the SNPs shown in Table 1.

Using such arrays or other kits/systems, the present invention provides methods of identifying the SNPs disclosed herein in a test sample. Such methods typically involve incubating a test sample of nucleic acids obtained from the patient with an array comprising one or more probes corresponding to at least one SNP position of the present invention, and assaying for binding of a nucleic acid from the test sample with one or more of the probes. Conditions for incubating a SNP detection reagent (or a kit/system that employs one or more such SNP detection reagents) with a test sample vary as briefly described hereinabove. Incubation conditions depend on such factors as the format employed in the assay, the detection methods employed, and the type and nature of the detection reagents used in the assay. One skilled in the art will recognize that any one of the commonly available hybridization, amplification and array assay formats can readily be adapted to detect the SNPs disclosed herein.

In the current invention, a test sample of a rheumatoid arthritis patient is analyzed for the presence of one or more SNPs of the invention. Typically, the test sample is a nucleic acid sample such as genomic DNA.

A SNP detection kit of the present invention may include components that are used to prepare nucleic acids from a test sample for the subsequent amplification and/or detection of a SNP-containing nucleic acid molecule. Such sample preparation components can be used to produce nucleic acid extracts (including DNA and/or RNA) from any bodily fluids (such as blood, serum, plasma, etc.) or from tissue samples. Methods of extracting nucleic acids, proteins, from samples are well known in the art and can be readily adapted to obtain a sample that is compatible with the system utilized. Automated sample preparation systems for extracting nucleic acids from a test sample are commercially available, and examples are Roche Molecular Systems' COBAS AmpliPrep System, Qiagen's BioRobot 9600, and Applied Biosystems' PRISM™ 6700 sample preparation system.

Microfluidic devices, which may also be referred to as “lab-on-a-chip” systems, biomedical micro-electro-mechanical systems (bioMEMs), or multicomponent integrated systems, are other examples of device or kits for analyzing SNPs. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more SNPs of the present invention. One example of a microfluidic system is disclosed in U.S. Pat. No. 5,589,136, which describes the integration of PCR amplification and capillary electrophoresis in chips. Exemplary microfluidic systems comprise a pattern of microchannels designed onto a glass, silicon, quartz, or plastic wafer included on a microchip. The movements of the samples may be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. See, for example, U.S. Pat. Nos. 6,153,073 and 6,156,181.

For genotyping SNPs, an exemplary microfluidic system may integrate, for example, nucleic acid amplification, primer extension, capillary electrophoresis, and a detection method such as laser induced fluorescence detection.

Correlating the Presence of SNP Biomarkers with Therapeutic Response

The present invention provides methods of SNP genotyping in evaluating the likelihood that a rheumatoid arthritis patient will respond to treatment with an IL-6R antibody, such as tocilizumab. Either female or male rheumatoid arthritis patients can be analyzed for the presence of one or more SNP biomarkers in accordance with the invention. The allelic frequencies may vary in particular populations. For some SNP biomarkers, white populations have the highest allele frequency of certain SNP biomarkers set forth in Table 1. In the context of this invention, the term “White” refers to an individual who reports himself or herself to be Caucasian or White.

The methods of the invention typically involve recording the presence or absence of a SNP biomarker associated with a beneficial therapeutic outcome, or in the alternative a negative therapeutic outcome, in a rheumatoid arthritis patient treated with an IL-6R antibody such as tocilizumab. This information may be stored in a computer readable form. Such a computer system typically comprises major subsystems such as a central processor, a system memory (typically RAM), an input/output (I/O) controller, an external device such as a display screen via a display adapter, serial ports, a keyboard, a fixed disk drive via a storage interface and the like. Many other devices can be connected, such as a network interface connected via a serial port.

The computer system also be linked to a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The computer system can comprise code for interpreting the results of a genotype study evaluating the presence of one or more SNP biomarkers. Thus in an exemplary embodiment, the genotype results are provided to a computer where a central processor executes a computer program for determining the propensity for a therapeutic response to treatment with an IL-6 receptor antibody.

The invention also provides the use of a computer system, such as that described above, which comprises: (1) a computer; (2) a stored bit pattern encoding the genotyping results obtained by the methods of the invention, which may be stored in the computer; (3) and, optionally, (4) a program for determining the likelihood for a positive therapeutic response, and/or the likelihood of a negative therapeutic outcome.

The invention further provides methods of generating a report based on the detection of the SNP pattern of a patient that has rheumatoid arthritis. Such a report is based on the detection of SNP alleles set forth in Table 1, or polymorphisms in strong linkage disequilibrium with the Table I SNP biomarker, that are associated with a therapeutic outcome. The report can make use of data that has been obtained from a patient sample where the data is available prior to the diagnosis of rheumatoid arthritis, e.g., the patient has been analyzed for SNP patterns prior to diagnosis. Thus, in some embodiments, a report is generated based on the SNP allele pattern of one or more SNP biomarkers set forth in Table 1 (or a SNP biomarker in strong linkage disequilibrium with the SNP biomarker) where the SNP alleles are determined in a sample from a patient who has been diagnosed with rheumatoid arthritis and is undergoing an evaluation for potential therapeutic efficacy of an IL-6 receptor antibody. In alternative embodiments, the report may make use of whole genome SNP array data for a rheumatoid arthritis patient where the data for that patient were generated previously, as the SNP biomarker pattern in genomic DNA is static.

A patient who is determined to have at least one SNP allele that is shown in Table 1 as having an association with a positive, i.e., improved, therapeutic endpoint, is considered to have an increased likelihood of exhibiting a positive therapeutic response to treatment with an IL-6R antibody, such as tocilizumab. Furthermore, patients having 2 or more of the SNP alleles, e.g., 3, 4, 5, 6, 7, 8, 9, or 10 or more SNP alleles (that are shown in Table 1 as having an association with a positive therapeutic endpoint) can have a further increased likelihood of exhibiting a positive therapeutic response.

Similarly, a patient who is determined to have at least one SNP allele that is shown in Table 1 as having an association with a negative therapeutic endpoint is considered to have an increased likelihood of having a negative therapeutic response to treatment with an IL-6R antibody, such as tocilizumab. Furthermore, patients having 2 or more of the SNP alleles, e.g., 3, 4, 5, 6, 7, 8, 9, or 10 or more SNP alleles, that are shown in Table 1 as having an association with a negative therapeutic endpoint, can have a further increased likelihood of exhibiting a negative therapeutic response to treatment with an IL-6R antibody such as tocilizumab.

In further embodiments, the methods of the invention can be employed to identify patients that are predicted to be non-responsive to treatment regimens comprising an IL-6R antibody such as tocilizumab. In Table 1, the beta or effect size (White(B)) can be either negative or positive. Thus, for example, the presence of a minor allele of a SNP may be associated with a negative outcome where the beta value for a rheumatoid arthritis symptom is positive.

In some embodiments, SNPs predicting both negative and positive outcomes may be combined into an algorithm to give an overall superior prediction of responsiveness to treatment.

### Example 1

### Identification of SNPs Associated with Therapeutic Response

This example identifies SNPs associated with the magnitude of response to treatment with tocilizumab in rheumatoid arthritis patients. The responses include safety end points: change in LDL at week 14 (ΔLDL) and Serious Infection Adverse Events (SIAEs).

706 DNA samples from TCZ treated patients from four tocilizumab (ACTEMRA®) Phase III trials were genotyped: The studies are as follows:

OPTION WA17822 (n=180) (Smolen et al, Lancet. 2008:371:987-997)—
TOWARD WA18063 (n=299) (Genovese et al Arthritis. Rheum. 2008; 58:2968-2980.),
RADIATE WA18062 (n=119) (Emery et al, Annals of the Rheumatic Diseases 2008; 67:1516-1523)
AMBITION WA17824 (n=108) (Jones. G et al, (2009) Ann Rheum Dis—ARD Online First, published on Mar. 17, 2009 as 10.1136/ard.2008.105197).
All of the four trials were randomized, multi-center double-blind, placebo-controlled studies to evaluate the efficacy and safety of tocilizumab in subjects with active rheumatoid arthritis.

AMBITION subjects, consenting to the genetics sub-study, were genotyped for >1 million markers genome-wide, using the Illumina 1M-Duo BeadChip. Subjects from the other trials were genotyped for >550,000 bi-allelic markers genome-wide using the Illumina HumanHap550v3_A array.

The aim of the current analysis was to detect genetic variants associated with baseline disease activity and response to tocilizumab in patients with rheumatoid arthritis. Associations with nine primary endpoints were investigated and reported.

Resultant data were statistically analyzed. Clinical endpoints used for analysis were:

Change from baseline (CFB) of Disease Activity Score (DAS28) at week 16 (ΔDAS28);

### Baseline DAS28;

CFB of Swollen Joint Count (SJC) at week 16;
CFB of Tender Joint Count (TJC) at week 16;
CFB of C-Reactive Protein (CRP) at week 8;
CFB of Health Assessment Questionnaire score (HAQ) at week 16;
CFB of Low-density lipoprotein (LDL) at week 14;

### Clearance; and

Infection adverse event.

All primary endpoints were subjected to adjusted single-point tests of association to Analysis Markers. Ordinary linear regression was applied to test for association between genetic markers and quantitative endpoints; logistic regression was applied to test association between genetic markers and dichotomous endpoints. All endpoints were also subject to multivariate analysis using Lasso variable selection (Wu & Lange, Annals Appl Stat 2: 224-244, 2008).

Two subject populations were considered. White subjects formed by far the largest racial group in this study, thus associations derived in those subjects, with the inherently low ethnic confounding, were analyzed. All subjects were adjusted by principal components from ancestry analysis (Price, et al., Nat Gen (38): 904-909).

The analysis identified 319 markers by the following criteria:

FDR q<0.05 in either subject set, or
p<1×10⁻⁵ in single-point analysis of White Subjects, or
p<1×10⁻⁴ in White subjects and a lower p-value in All, or
selected by Lasso analysis of either subject set

The thresholds used for identification are non-conservative; they reflect a greater emphasis on false negative rates than on false positive rates. The threshold of p<10⁻⁵ however, has been used in other studies in this field. For example, it was used for reporting in the National Human Genome Research Institute's (NHGRI) Catalogue of Published Genome-Wide Association Studies (Hindorff et al, 2008). A number of markers with p<1×10⁻⁷ (genome-wide significance) or FDR q<0.05 in either White Subjects or the All subjects set are identified.

Table 1 provides a list of 319 biomarkers identified. In Table 1, “BP” refers to the base pair position on the chromosome; “MAF” refers to “Major Allele Frequency;” “White (B)” refers to the ‘Beta’ or ‘coefficient’ and describes the effect size of the response; the “C” preceding the terms in the “Trait” filed refers to “Change”.

In this study, departure from Hardy-Weinberg Equilibrium (HWE) is used to flag potential genotyping error or population admixture. A test for Hardy-Weinberg Equilibrium was performed on all SNPs. Among the 319 SNPs in Table 1, with the exception of 4 SNPs, all had a HWE p-value of greater than 0.02, indicating that there was no significant departure from HWE. The 4 SNPs with lower p-values are rs3769124 (0.0024), rs6519366 (0.0065), rs2330191 (0.0086) and rs715594 (0.0086). The Hardy-Weinberg deviation test p-values for these 4 SNPs are considered as false positives at the genome-wide scale level.

The term “White (B)” in Table 1 refers to the ‘Beta’ or ‘coefficient’ and describes the effect size. The sign of beta indicates the direction of the effect, i.e., a positive number indicates an increase in units of a particular endpoint and a negative value indicates a reduction. For example, SNP number 8 in Table 1, rs11205321, is significant for the clinical endpoint CTJC_WK16 and has a coefficient or beta of 5.033. This indicates that the mean change for every copy of the minor allele is approx +5 TJC units relative to the patient group that doesn't carry the minor allele. As greater TJC means poor response, the minor allele is the risk allele for poorer response for this SNP. Thus, using the example rs11205321, the presence of the minor allele is associated with an increase in TJC units, i.e., is associated with a negative therapeutic endpoint (increased TJC units); or equivalently, the presence of the major allele is associated with better response, with mean change for each copy of the major allele equals to −5 TJC unit relative to the patient group homozygous of the minor allele. Accordingly, a patient carrying the minor allele (2 copies or at least one copy, depending on the further assessment) will be excluded from treatment.

Two hundred and ninety-one unique markers were highlighted overall; these were either nominally significant in single-point analysis, or retained in a multivariate Lasso model. Markers identified included one marker (rs6004913) associated to three efficacy endpoints and six markers (rs8049145, rs9883073, rs4074617, rs7104941, rs11886534 and rs7521783) associated to two efficacy endpoints. Marker rs6004913 is in linkage disequilibrium with rs2236006, a non-synonymous coding change in MYO18B (MYOSIN XVIIIB). Marker rs7104941 is an intronic marker in DKK3 (Dickkopf Homolog 3). The rest of the SNPs in Table 2 are intergenic.

The seven markers noted below are shown in Table 2:

TABLE 2

| Marker | | | | | | | | |
|---|---|---|---|---|---|---|---|---|
| Endpoints | Chr | BP | Coding | CDAS28 | CSJC | CTJC | CHAQ | No. |
| rs7521783 | 1 | 148409817 | — | 4.02e−05 | | 7.97e−06 | | 2 |
| rs11886534 | 2 | 3815178 | — | 5.40e−05 | 5.39e−08 | | | 2 |
| rs9883073 | 3 | 41259540 | — | | 0.000342 | 0.001194 | | 2 |
| rs4074617 | 6 | 3858629 | — | | | 8.18e−05 | 0.000107 | 2 |
| rs7104941 | 11 | 11977192 | DKK3 | | | 2.39e−05 | 1.88e−06 | 2 |
| rs8049145 | 16 | 17060587 | — | | 4.84e−05 | 0.000189 | | 2 |
| rs6004913 | 22 | 24768495 | — | 3.94e−06 | | 9.71e−06 | 1.50e−06 | 3 |

One marker was highlighted three times: rs6004913 was identified for Change in DAS28, Change in Tender Joint Count and Change in HAQ Score. It is an intergenic SNP in LD (r2=0.62) with a non-synonymous coding change (rs2236005) in MYO18B. This is a putative tumor suppressor gene that may regulate anchorage independent cell growth

Only one of the 7 markers from Table 2 fell with a gene; rs7104941 is an intronic marker in DKK3 (Dickkopf-related protein 3 precursor). The secreted protein interacts with the Wnt signaling pathway.

Marker rs7521783 is in strong LD (r²=0.95) with two intronic markers in PLEKHO1 (pleckstrin homology domain containing, family O member 1). The protein produced plays a role in the regulation of the actin cytoskeleton through its interactions with actin capping protein. It has been implicated in the promotion of apoptosis induced by tumor necrosis factor TNF. The marker is also in strong LD (r² range: 0.65-0.95) with 20 intronic markers and one marker in the 5′ UTR of VPS45 (vacuolar protein sorting-associated protein 45). The protein plays an important role in the segregation of intracellular molecules into distinct organelles. High expression of the gene in peripheral blood mononuclear cells suggests a role in trafficking proteins, including inflammatory mediators. Finally, the marker is also in lower LD with markers in C1orf54, MRPS21 (mitochondrial 28S ribosomal protein S21), PRPF3 (U4/U6 small nu-clear ribonucleoprotin PRP3), KIAA0460 (uncharacterised protein), TARS2 (threonyl-tRNA synthetase, mitochondrial precursor), and ECM1 (extracellular matrix protein 1 precursor).

Marker rs11886534 is in LD (r²=0.45) with an intronic marker in AC019172.4, a putative double-cortin domain-containing protein.

Marker rs9883073 is in perfect LD with 7 intronic markers in CTNNB1 (Catenin beta-1). CTNNB1 is involved in the regulation of cell adhesion and in signal transduction through the Wnt pathway. The marker is also in LD (r²=0.97) with an intronic marker in ULK4, a serine/threonine-protein kinase.

Marker rs4074617 is intergenic and not in LD with any gene-based markers using Hapmap CEU data.

Lastly, marker rs8049145 is another intergenic SNP. It is in LD (r²=0.534) with a marker in the 3′ UTR of XYLT1 (xylosyltransferase 1). XYLT1 is required for the biosynthesis of the glycosaminoglycan chains characteristic of proteoglycans.

Rs6078937 lies within an intron of the SPTLC3 gene (Serine Palmitoyltransferase, Long Chain base subunit 3). This SNP was identified from the analysis of cDAS28 and is the only marker highlighted by all four analysis criteria for a specific clinical endpoint:

### P=4.43×10⁻⁶ in White Subjects

### P=1.73×10⁻⁶ in All Subjects

### Selected by Lasso in White Subjects

### Selected by Lasso in All Subjects.

SPTLC3 is part of the trimeric serine palmitoyltransferase (SPT) complex and catalyses the rate-limiting step of the de novo synthesis of sphingolipids. Sphingolipids play a role in inflammatory response and in the regulation of TNF-alpha.

All of the 319 SNPs set forth in Table 1 may be used as univariate biomarkers or in combination in a multivariate model.

### Example 2

### SNP Alleles for the Detection of Patient Response to Treatment with Tocilizumab

The SNP rs6004913 or any neighboring SNPs in tight correlation, i.e., linkage disequilibrium, with it, may be used alone or in combination with additional genetic or other non-genetic variables in a composite biomarker score that allows the individual prediction of response to tocilizumab prior to treatment. In a such a score, the effect of rs6004913 may be additive, dominant or recessive. The clinical response is defined based on change of DAS score, Eular criteria or ACR20, ACR50 or ACR70. Additional covariates included in the composite biomarker score can include, but are not limited to, platelet counts, serum IL-6 level at baseline, HLA-DRB1 genotypes. The functional form of the biomarker score may be linear or on-linear, including interaction term between covariates or tree-based.

The SNP rs6078937 or any neighboring SNPs in tight correlation, i.e., linkage disequilibrium, with it, may be used alone or in combination with additional genetic variables, including SNP rs6004913, or other non-genetic variables in a composite biomarker score that allows the individual prediction of response to tocilizumab prior to treatment. In a such a score, the effect of rs6004913 may be additive, dominant or recessive. The clinical response may be defined based on change of DAS score, Eular criteria or ACR20, ACR50 or ACR70. Additional covariates included in the composite biomarker score can include, but are not limited to, platelet counts, serum IL-6 level at baseline, HLA-DRB1 genotypes. The functional form of the biomarker score may be linear or non-linear, including interaction term between covariates or tree-based.

It is understood that the examples and embodiments described herein are for illustrative purposes only and that various modifications or changes in light thereof will be suggested to persons skilled in the art and are to be included within the spirit and purview of this application and scope of the appended claims.

All publications, patents, accession number, and patent applications cited herein are hereby incorporated by reference in their entirety for all purposes.

TABLE 1

| Chr | SNP | BP | MAF* | White(B) | White(p) | All(p) | Coding |
|---|---|---|---|---|---|---|---|
| 1 | rs6429673 | 14898561 | 0.1225 | −0.187 | 9.85E−05 | 1.51E−05 | AB028949 A |
| 1 | rs1891419 | 31124100 | 0.2935 | −0.1523 | 1.12E−05 | 0.0004 | SDC3 |
| 1 | rs11578253 | 77037648 | 0.4258 | 4.604 | 0.0004 | 0.0004 | — |
| 1 | rs10873941 | 77675569 | 0.1672 | 0.1291 | 2.36E−06 | 4.67E−05 | AK5 |
| 1 | rs12401737 | 111631018 | 0.4468 | −0.1142 | 0.0001 | 0.0054 | — |
| 1 | rs7555330 | 111728687 | 0.4244 | −1.892 | 0.0002 | 0.002 | — |
| 1 | rs12063329 | 148269060 | 0.1597 | 5.033 | 1.44E−05 | 6.92E−07 | — |
| 1 | rs11205321 | 148292831 | 0.1603 | 5.033 | 1.44E−05 | 1.72E−06 | — |
| 1 | rs7521783 | 148409817 | 0.1648 | 0.5222 | 4.02E−05 | 9.98E−06 | — |
| 1 | rs7521783 | 148409817 | 0.1648 | 5.083 | 7.97E−06 | 2.29E−06 | — |
| 1 | rs1882926 | 184678202 | 0.1298 | 3.241 | 2.43E−05 | 4.22E−06 | — |
| 1 | rs6672638 | 184684107 | 0.1315 | 3.241 | 2.43E−05 | 1.86E−05 | PDC |
| 1 | rs12062743 | 184714214 | 0.1321 | 3.13 | 4.51E−05 | 2.58E−05 | — |
| 1 | rs7542810 | 184798854 | 0.1288 | 3.146 | 4.48E−05 | 5.84E−06 | — |
| 1 | rs1569879 | 184814575 | 0.1287 | 3.139 | 4.57E−05 | 9.01E−06 | — |
| 1 | rs12405141 | 184890093 | 0.1332 | 3.232 | 2.12E−05 | 3.04E−06 | — |
| 1 | rs10911901 | 184890629 | 0.1531 | 3.116 | 1.42E−05 | 1.03E−05 | — |
| 1 | rs10911905 | 184919018 | 0.1366 | 3.14 | 3.24E−05 | 2.23E−05 | — |
| 1 | rs2143417 | 184920410 | 0.1576 | 3.024 | 2.37E−05 | 3.65E−06 | — |
| 1 | rs2383529 | 184935715 | 0.215 | 2.661 | 1.74E−05 | 7.19E−06 | — |
| 1 | rs12401885 | 184949498 | 0.1426 | 3.099 | 3.63E−05 | 3.57E−05 | — |
| 1 | rs3892225 | 216619920 | 0.1608 | 0.1668 | 5.11E−05 | 3.41E−05 | M19154 TG [symbol] |
| 1 | rs1266380 | 234748373 | 0.4955 | −0.105 | 0.0005 | 0.0025 | — |
| 1 | rs6429165 | 238126680 | 0.2141 | 0.2271 | 4.96E−05 | 6.27E−06 | — |
| 2 | rs7568276 | 2524085 | 0.3273 | −0.1364 | 3.31E−05 | 2.69E−05 | — |
| 2 | rs17018026 | 3814635 | 0.3218 | −2.236 | 3.48E−05 | 3.42E−05 | — |
| 2 | rs11886534 | 3815178 | 0.4385 | −0.3627 | 5.40E−05 | 7.87E−07 | — |
| 2 | rs11886534 | 3815178 | 0.4385 | −2.793 | 5.39E−08 | 1.38E−07 | — |
| 2 | rs13010129 | 5354426 | 0.4391 | −0.1678 | 8.31E−05 | 0.0002 | — |
| 2 | rs2349683 | 5359622 | 0.2203 | −0.2235 | 8.98E−06 | 1.85E−06 | — |
| 2 | rs4669069 | 6488209 | 0.4881 | 0.0766 | 0.0001 | 8.32E−05 | — |
| 2 | rs10188894 | 6561184 | 0.4921 | 0.2552 | 0.0047 | 0.023 | — |
| 2 | rs11694489 | 10380303 | 0.078 | −0.2521 | 5.81E−06 | 0.0001 | — |
| 2 | rs16856119 | 10385274 | 0.079 | −0.2558 | 3.58E−06 | 4.65E−06 | — |
| 2 | rs798369 | 16800799 | 0.4441 | −0.0826 | 4.41E−05 | 0.0002 | — |
| 2 | rs703297 | 19489299 | 0.4814 | 1.603 | 0.0003 | 0.0003 | — |
| 2 | rs721064 | 21371947 | 0.3284 | 2.413 | 2.63E−05 | 1.11E−05 | — |
| 2 | rs1971201 | 52857244 | 0.4807 | 0.398 | 1.04E−05 | 8.73E−06 | — |
| 2 | rs7577197 | 53028460 | 0.4418 | −0.4459 | 9.09E−07 | 2.52E−06 | — |
| 2 | rs892866 | 79263855 | 0.0988 | 0.1091 | 0.4261 | 0.8977 | — |
| 2 | rs7557391 | 85171226 | 0.4763 | −0.0373 | 0.6464 | 0.7616 | — |
| 2 | rs7603857 | 122711103 | 0.447 | 0.2199 | 0.0023 | 0.0012 | — |
| 2 | rs10199719 | 148875899 | 0.4836 | −0.1835 | 0.0001 | 0.0015 | — |
| 2 | rs11888208 | 167021697 | 0.1377 | 5.485 | 4.98E−06 | 0.0004 | SCN7A |
| 2 | rs13017982 | 167034898 | 0.1383 | 5.485 | 4.98E−06 | 0.0004 | SCN7A |
| 2 | rs2176592 | 170891256 | 0.3459 | 0.216 | 4.97E−05 | 1.57E−05 | AF391554 A |
| 2 | rs2139133 | 170902163 | 0.4024 | 0.1979 | 9.34E−05 | 4.24E−05 | AF391554 A |
| 2 | rs3112943 | 180210601 | 0.4769 | −0.0663 | 0.026 | 0.0017 | ZNF533 |
| 2 | rs3771074 | 187993955 | 0.0215 | −0.5805 | 7.37E−05 | 1.72E−05 | — |
| 2 | rs13008446 | 200336745 | 0.333 | 0.2259 | 7.60E−06 | 6.95E−05 | — |
| 2 | rs1429890 | 209216592 | 0.4385 | 0.153 | 0.0003 | 7.20E−06 | — |
| 2 | rs1402766 | 212741546 | 0.3787 | 0.0925 | 0.0355 | 0.0001 | ERBB4 |
| 2 | rs3739070 | 238971007 | 0.0887 | 0.1642 | 4.49E−06 | 6.35E−05 | AF242456 T |
| 2 | rs3769124 | 239014101 | 0.1275 | 0.1259 | 7.12E−06 | 0.0004 | ASB1 |
| 3 | rs1353825 | 1313259 | 0.2534 | 0.1056 | 0.2591 | 0.2267 | CNTN6 |
| 3 | rs13064317 | 2388859 | 0.0557 | 0.8835 | 1.09E−06 | 0.0008 | — |
| 3 | rs6442763 | 2896515 | 0.4419 | −0.0763 | 6.65E−05 | 4.64E−05 | CNTN4 |
| 3 | rs9852580 | 3008170 | 0.3036 | 1.948 | 1.29E−05 | 0.0002 | CNTN4 |
| 3 | rs163577 | 3075787 | 0.1258 | −0.2587 | 9.96E−05 | 4.58E−05 | — |
| 3 | rs6763225 | 20390516 | 0.2054 | −0.1526 | 6.01E−05 | 1.78E−05 | — |
| 3 | rs12152385 | 21671143 | 0.0809 | 0.2064 | 0.1599 | 0.1697 | ZNF659 |
| 3 | rs6550967 | 25371764 | 0.3505 | −1.977 | 0.0004 | 0.0001 | — |
| 3 | rs1488248 | 26768522 | 0.4621 | −0.187 | 0.0001 | 3.79E−05 | — |
| 3 | rs4973821 | 26826361 | 0.4045 | −0.1631 | 0.0011 | 5.08E−05 | — |
| 3 | rs11924987 | 30299347 | 0.4278 | 2.824 | 0.0003 | 8.83E−05 | — |
| 3 | rs9883073 | 41259540 | 0.4639 | −1.801 | 0.0003 | 0.0011 | — |
| 3 | rs9883073 | 41259540 | 0.4639 | −2.557 | 0.0012 | 0.0105 | — |
| 3 | rs1013758 | 43601379 | 0.4368 | −0.0781 | 0.0134 | 0.0029 | TMEM16K F |
| 3 | rs11916112 | 56899751 | 0.2376 | 0.005 | 0.9567 | 0.937 | AF433662 |
| 3 | rs930567 | 106835835 | 0.4195 | −0.1858 | 7.93E−06 | 3.67E−05 | — |
| 3 | rs12631538 | 117984668 | 0.4921 | 1.886 | 0.0003 | 0.0004 | — |
| 3 | rs3845918 | 123418536 | 0.2573 | −3.508 | 0.0001 | 0.0005 | — |
| 3 | rs4854785 | 135200638 | 0.2122 | 4.975 | 1.88E−05 | 2.54E−05 | SLCO2A1 |
| 3 | rs4627 | 144009284 | 0.1862 | 4.193 | 9.03E−05 | 1.18E−05 | — |
| 3 | rs12490517 | 163936122 | 0.3318 | −0.32 | 0.0013 | 1.45E−05 | — |
| 3 | rs16825529 | 176550912 | 0.47 | 0.3242 | 0.0004 | 0.0003 | NAALADL2 |
| 3 | rs9812934 | 177492211 | 0.4577 | −0.1512 | 0.0003 | 2.52E−05 | — |
| 3 | rs6444188 | 188160630 | 0.3657 | 1.778 | 4.74E−05 | 4.18E−05 | — |
| 3 | rs3856776 | 190781102 | 0.4949 | −0.0568 | 0.0046 | 0.0002 | — |
| 4 | rs1503990 | 21260718 | 0.4707 | 0.0698 | 0.0003 | 0.0003 | — |
| 4 | rs13116880 | 54654762 | 0.1337 | 0.5536 | 2.50E−06 | 3.23E−06 | AY229892 |
| 4 | rs3762885 | 83524302 | 0.2223 | −3.774 | 7.69E−05 | 4.53E−06 | — |
| 4 | rs13130041 | 88448163 | 0.2709 | 0.125 | 0.0003 | 1.61E−05 | AY186250 H |
| 4 | rs1907101 | 157544838 | 0.2511 | 0.5008 | 4.78E−06 | 0.0002 | — |
| 4 | rs12512817 | 178208836 | 0.0801 | 0.6862 | 3.58E−06 | 0.0002 | — |
| 5 | rs2132538 | 7169396 | 0.1467 | 0.4824 | 7.21E−05 | 3.45E−06 | — |
| 5 | rs1496361 | 7189893 | 0.1309 | 0.5555 | 1.15E−05 | 8.51E−07 | — |
| 5 | rs4702429 | 7190379 | 0.1311 | 0.556 | 1.16E−05 | 5.37E−07 | — |
| 5 | rs1390568 | 18607264 | 0.4543 | 0.1868 | 1.24E−05 | 7.14E−06 | — |
| 5 | rs1390566 | 18660238 | 0.3078 | 0.1952 | 1.80E−05 | 2.24E−06 | — |
| 5 | rs10054604 | 18728628 | 0.2669 | 0.1892 | 9.75E−05 | 7.87E−05 | — |
| 5 | rs954478 | 25716149 | 0.3341 | −0.0782 | 0.0002 | 0.0008 | — |
| 5 | rs10941110 | 34034836 | 0.478 | 0.0379 | 0.6355 | 0.715 | AMACR AY3 |
| 5 | rs6896511 | 67843968 | 0.4056 | 2.34 | 1.89E−05 | 1.41E−05 | — |
| 5 | rs17476297 | 85945880 | 0.149 | 0.2001 | 5.53E−06 | 2.78E−05 | — |
| 5 | rs12153530 | 95843260 | 0.2135 | −0.116 | 8.10E−06 | 0.0008 | — |
| 5 | rs1586726 | 108938984 | 0.0869 | 0.3614 | 4.52E−05 | 7.28E−06 | — |
| 5 | rs6873898 | 108950067 | 0.0869 | 0.3614 | 4.52E−05 | 7.70E−06 | — |
| 5 | rs7735853 | 120422853 | 0.105 | 0.0574 | 0.6577 | 0.9285 | — |
| 5 | rs11242396 | 137028864 | 0.2709 | −0.3676 | 9.86E−05 | 4.85E−05 | KLHL3 BC03 [symbol] |
| 5 | rs10037652 | 153827061 | 0.3685 | −0.0588 | 0.0044 | 0.0004 | — |
| 5 | rs3914991 | 162107732 | 0.4938 | 0.5418 | 8.27E−06 | 1.13E−05 | — |
| 5 | rs703505 | 169741980 | 0.3922 | −0.1221 | 0.0001 | 2.09E−05 | KCNMB1 |
| 6 | rs4074617 | 3858629 | 0.496 | 0.1198 | 0.0001 | 0.0002 | — |
| 6 | rs4074617 | 3858629 | 0.496 | 3.197 | 8.18E−05 | 0.001 | — |
| 6 | rs10484340 | 14765721 | 0.4165 | 7.439 | 5.16E−05 | 0.0001 | — |
| 6 | rs3734557 | 40468443 | 0.3911 | −1.36 | 0.0085 | 0.0259 | LRFN2 |
| 6 | rs4714432 | 41279292 | 0.4379 | 6.456 | 0.0001 | 7.96E−05 | — |
| 6 | rs10485252 | 70051082 | 0.3527 | 1.772 | 5.38E−05 | 4.55E−06 | BAI3 |
| 6 | rs6910395 | 75408225 | 0.0491 | 0.8939 | 2.21E−05 | 4.55E−06 | — |
| 6 | rs1088022 | 82197030 | 0.4972 | 1.641 | 0.0001 | 0.0039 | — |
| 6 | rs9294310 | 85491994 | 0.0378 | 5.469 | 2.11E−05 | 1.50E−05 | — |
| 6 | rs7755116 | 100908533 | 0.4434 | 0.131 | 0.0009 | 0.0008 | — |
| 6 | rs12213919 | 152528065 | 0.1326 | 0.2524 | 3.31E−05 | 1.40E−05 | SYNE1 AF44 |
| 6 | rs9295134 | 161370518 | 0.1146 | 0.4831 | 5.30E−05 | 4.51E−07 | MAP3K4 |
| 6 | rs12110787 | 161394956 | 0.0993 | 0.5366 | 3.02E−05 | 3.03E−08 | MAP3K4 |
| 6 | rs9355870 | 161415648 | 0.1123 | 0.4737 | 9.30E−05 | 4.28E−06 | MAP3K4 |
| 6 | rs634651 | 165885469 | 0.3153 | −0.4141 | 2.68E−05 | 0.0008 | PDE10A AF1 |
| 7 | rs6972567 | 8276518 | 0.4473 | −3.631 | 6.81E−06 | 5.62E−05 | — |
| 7 | rs2053380 | 12367362 | 0.2872 | 0.2195 | 3.88E−06 | 0.0003 | AK027618 |
| 7 | rs2041582 | 17712578 | 0.3486 | −0.2116 | 3.50E−05 | 0.0001 | — |
| 7 | rs6461389 | 18873555 | 0.3567 | −0.1743 | 0.0004 | 4.87E−05 | AJ459808 H [symbol] |
| 7 | rs2529753 | 20805476 | 0.3962 | 0.1094 | 0.0006 | 0.0102 | — |
| 7 | rs7805619 | 31297906 | 0.2889 | −0.3597 | 3.24E−05 | 1.49E−05 | — |
| 7 | rs10270760 | 51799720 | 0.0102 | 1.754 | 7.18E−06 | 0.0161 | — |
| 7 | rs1403155 | 67688349 | 0.0164 | 1.567 | 3.78E−07 | 0.0452 | — |
| 7 | rs9647718 | 73577329 | 0.1964 | 0.2088 | 6.43E−05 | 1.71E−06 | GTF2IRD1 |
| 7 | rs3801469 | 73578546 | 0.2235 | 0.1991 | 7.17E−05 | 1.60E−05 | GTF2IRD1 |
| 7 | rs17156280 | 81881926 | 0.0152 | −0.1978 | 0.5479 | 0.7912 | CACNA2D1 |
| 7 | rs1209001 | 91278295 | 0.3166 | 0.3881 | 5.11E−05 | 4.46E−05 | — |
| 7 | rs10249873 | 91663104 | 0.315 | 0.3803 | 6.85E−05 | 6.39E−05 | — |
| 7 | rs2724041 | 95914863 | 0.4394 | 0.1643 | 0.0008 | 0.0002 | — |
| 7 | rs562221 | 105078680 | 0.4588 | 0.1619 | 0.0001 | 0.0019 | — |
| 7 | rs10215964 | 129033356 | 0.0837 | 0.29 | 0.0571 | 0.01 | — |
| 7 | rs10954644 | 138627746 | 0.0367 | 1.082 | 1.44E−06 | 0.1042 | AK125956 A |
| 7 | rs10254641 | 147536373 | 0.4938 | 1.34 | 0.0104 | 0.0003 | CNTNAP2 |
| 8 | rs10108210 | 2588854 | 0.4588 | 0.0746 | 0.0136 | 0.0035 | — |
| 8 | rs11250154 | 11534095 | 0.2822 | 0.1991 | 4.38E−05 | 1.10E−05 | — |
| 8 | rs7013759 | 26624548 | 0.0452 | 0.201 | 4.00E−05 | 6.33E−06 | — |
| 8 | rs6557943 | 26629334 | 0.0431 | 0.2015 | 4.83E−05 | 7.31E−06 | — |
| 8 | rs1119176 | 52283079 | 0.3869 | −0.0796 | 0.0001 | 2.61E−05 | — |
| 8 | rs10957545 | 72381472 | 0.4227 | −2.61 | 0.001 | 0.0024 | EYA1 |
| 8 | rs2045506 | 87936463 | 0.4396 | −0.1779 | 0.0002 | 0.0132 | — |
| 8 | rs1431884 | 98248154 | 0.0554 | 7.09 | 5.83E−05 | 1.07E−05 | — |
| 8 | rs7830351 | 107118001 | 0.0976 | 0.6292 | 7.48E−05 | 1.32E−05 | — |
| 8 | rs12544984 | 126531252 | 0.0583 | 8.316 | 3.75E−06 | 0.0012 | — |
| 8 | rs10808556 | 128482329 | 0.4041 | 3.182 | 8.96E−05 | 0.0003 | — |
| 8 | rs7828882 | 137471530 | 0.0011 | 39.35 | 1.89E−06 | 0.5385 | — |
| 9 | rs10122609 | 9273557 | 0.1507 | −0.1808 | 4.85E−05 | 3.48E−06 | — |
| 9 | rs992457 | 13695882 | 0.4024 | −0.0615 | 0.4548 | 0.0922 | — |
| 9 | rs7018762 | 14412288 | 0.3584 | 2.542 | 0.0025 | 0.0008 | — |
| 9 | rs1887444 | 18942509 | 0.3149 | 0.1555 | 0.0025 | 6.14E−05 | C9orf138 |
| 9 | rs3900585 | 31302460 | 0.0096 | 31.27 | 3.83E−06 | 1.55E−05 | — |
| 9 | rs10114881 | 75865891 | 0.3132 | −0.1226 | 0.0002 | 4.31E−05 | — |
| 9 | rs914365 | 77913127 | 0.4317 | 1.738 | 0.0014 | 0.0157 | PCSK5 |
| 9 | rs4467996 | 93557536 | 0.4103 | 4.744 | 0.0002 | 0.0001 | BC033697 R |
| 9 | rs12377425 | 102982369 | 0.3962 | −0.0794 | 6.61E−05 | 0.0005 | — |
| 9 | rs13302591 | 132054458 | 0.1563 | 0.567 | 8.20E−08 | 9.78E−06 | — |
| 9 | rs7045878 | 132059019 | 0.3237 | 0.3511 | 6.32E−05 | 6.10E−06 | — |
| 10 | rs4880930 | 1860359 | 0.0265 | 16.5 | 8.44E−06 | 1.13E−05 | — |
| 10 | rs7086455 | 16623255 | 0.2037 | 0.477 | 2.17E−06 | 0.0002 | — |
| 10 | rs2789310 | 19368441 | 0.0852 | 0.5912 | 5.97E−05 | 5.52E−05 | — |
| 10 | rs2339893 | 53358303 | 0.2893 | −1.918 | 0.0007 | 0.0006 | D45864 PRK |
| 10 | rs11000178 | 53362087 | 0.2867 | −1.932 | 0.0006 | 0.0005 | D45864 PRK |
| 10 | rs996320 | 55671084 | 0.2906 | −0.0609 | 0.0052 | 0.0001 | PCDH15 AL [symbol] |
| 10 | rs1219862 | 55860311 | 0.3248 | −0.0714 | 0.001 | 0.0002 | PCDH15 AL [symbol] |
| 10 | rs7077638 | 67997021 | 0.4633 | 0.0437 | 0.6064 | 0.6993 | CTNNA3 BC [symbol] |
| 10 | rs7923078 | 68100834 | 0.1196 | −0.1474 | 0.2163 | 0.0856 | CTNNA3 BC [symbol] |
| 10 | rs1471824 | 72405746 | 0.4944 | 0.0855 | 2.28E−05 | 0.0002 | — |
| 10 | rs1267723 | 76795860 | 0.4096 | 0.4159 | 6.54E−06 | 4.55E−06 | — |
| 10 | rs1259590 | 76798006 | 0.4266 | 0.3976 | 1.26E−05 | 3.90E−06 | — |
| 10 | rs2673474 | 79081451 | 0.2635 | 2.412 | 8.93E−05 | 8.19E−06 | — |
| 10 | rs2670109 | 79083665 | 0.2945 | 2.535 | 2.57E−05 | 2.49E−06 | — |
| 10 | rs2254094 | 79091250 | 0.2869 | 2.443 | 4.46E−05 | 4.18E−06 | — |
| 10 | rs12416404 | 84675198 | 0.285 | −0.3826 | 6.07E−05 | 5.61E−05 | NRG3 |
| 10 | rs12243526 | 121998220 | 0.3347 | −0.134 | 6.20E−05 | 1.75E−05 | — |
| 10 | rs10886648 | 122005564 | 0.3392 | −0.1401 | 2.88E−05 | 3.76E−06 | — |
| 10 | rs10830067 | 129137470 | 0.4667 | 1.518 | 0.0018 | 0.0011 | DOCK1 |
| 10 | rs9419387 | 133608348 | 0.3476 | 0.6549 | 0.0016 | 0.0001 | BC047379 |
| 11 | rs1554519 | 10860643 | 0.4187 | −1.261 | 0.014 | 0.024 | — |
| 11 | rs4910008 | 11436442 | 0.4682 | −3.024 | 0.0002 | 0.0018 | GALNTL4 |
| 11 | rs7104941 | 11977192 | 0.1834 | −0.1894 | 1.88E−06 | 1.00E−05 | DKK3 |
| 11 | rs7104941 | 11977192 | 0.1834 | −4.384 | 2.39E−05 | 9.04E−07 | DKK3 |
| 11 | rs3750940 | 11979474 | 0.1972 | −0.1849 | 2.01E−06 | 4.96E−06 | DKK3 |
| 11 | rs4500466 | 11998072 | 0.3397 | −0.1324 | 7.59E−05 | 2.03E−05 | — |
| 11 | rs11227306 | 65335248 | 0.3678 | 0.1249 | 7.73E−05 | 2.09E−05 | — |
| 11 | rs1892952 | 76042374 | 0.4989 | −1.842 | 0.0003 | 0.0002 | — |
| 11 | rs1461687 | 98598979 | 0.4588 | −2.061 | 6.02E−05 | 2.45E−05 | — |
| 11 | rs2165662 | 99647982 | 0.4769 | 0.1755 | 0.0002 | 3.71E−05 | CNTN5 |
| 11 | rs12271603 | 104617228 | 0.4712 | −0.1116 | 0.0002 | 6.92E−05 | — |
| 11 | rs509305 | 109258743 | 0.0011 | 0.9277 | 5.75E−06 | 0.0151 | — |
| 11 | rs7126496 | 109306304 | 0.0011 | 0.9277 | 5.75E−06 | 0.0063 | — |
| 11 | rs378331 | 113599150 | 0.377 | 0.3163 | 0.0004 | 0.0122 | ZBTB16 |
| 11 | rs638000 | 119391124 | 0.4142 | −0.179 | 3.75E−05 | 0.0005 | — |
| 11 | rs10750523 | 132391583 | 0.1823 | −0.8388 | 0.2174 | 0.1323 | OPCML |
| 11 | rs570113 | 133627460 | 0.3172 | −0.131 | 9.67E−05 | 5.35E−05 | THY28 THY [symbol] |
| 12 | rs1860393 | 3309218 | 0.4904 | 0.1235 | 0.0102 | 0.0002 | — |
| 12 | rs4316580 | 4782296 | 0.1271 | −0.0994 | 0.4106 | 0.668 | — |
| 12 | rs1560011 | 9714219 | 0.4311 | 0.6587 | 0.0013 | 0.0004 | CLEC2D BC0 |
| 12 | rs11052384 | 9725748 | 0.1714 | 0.5225 | 5.89E−05 | 4.98E−05 | CLEC2D BC0 |
| 12 | rs7295036 | 24548814 | 0.3995 | 0.0672 | 0.0011 | 2.73E−05 | — |
| 12 | rs17099183 | 61912959 | 0.092 | 0.6366 | 3.75E−06 | 3.72E−05 | — |
| 12 | rs1913134 | 84279990 | 0.209 | 2.103 | 4.88E−05 | 2.46E−05 | — |
| 12 | rs11104481 | 86353732 | 0.0666 | −6.408 | 9.00E−05 | 4.43E−05 | — |
| 12 | rs406117 | 91323127 | 0.3083 | −0.1962 | 0.0002 | 1.33E−05 | — |
| 12 | rs12580153 | 91342613 | 0.2861 | −0.2155 | 6.97E−05 | 3.68E−06 | AJ845168 C [symbol] |
| 12 | rs11066740 | 112716731 | 0.1096 | 0.645 | 1.56E−06 | 0.0003 | — |
| 12 | rs7314138 | 130331640 | 0.4419 | 0.1856 | 0.0002 | 0.0001 | — |
| 13 | rs9508345 | 28783981 | 0.4825 | −0.0992 | 0.225 | 0.5834 | — |
| 13 | rs9594987 | 43128994 | 0.4266 | −0.1125 | 0.0002 | 1.56E−05 | — |
| 13 | rs9534561 | 46650887 | 0.4374 | 0.0826 | 3.28E−05 | 0.0003 | — |
| 13 | rs1832696 | 54005651 | 0.4481 | 0.1906 | 0.0001 | 0.0118 | — |
| 13 | rs7988602 | 64120863 | 0.0971 | −0.6868 | 5.40E−06 | 0.0005 | — |
| 13 | rs9529287 | 67395625 | 0.4295 | 6.298 | 8.74E−05 | 5.79E−05 | — |
| 13 | rs9600195 | 73437526 | 0.0102 | 36.17 | 5.02E−07 | 2.28E−06 | KLF12 |
| 13 | rs7994999 | 84040671 | 0.0011 | 36.64 | 1.23E−05 | 7.54E−06 | — |
| 13 | rs722952 | 85128914 | 0.4746 | 0.1443 | 0.0006 | 8.80E−06 | — |
| 13 | rs7337223 | 90616096 | 0.0316 | 0.8679 | 4.25E−05 | 9.11E−06 | — |
| 13 | rs7984041 | 90618775 | 0.0316 | 0.8679 | 4.25E−05 | 9.11E−06 | — |
| 13 | rs2282423 | 96586295 | 0.237 | 0.4564 | 1.76E−06 | 0.0008 | — |
| 13 | rs3902523 | 96635106 | 0.1693 | 0.4865 | 4.73E−06 | 0.0002 | — |
| 13 | rs4771267 | 96639269 | 0.2161 | 0.473 | 1.52E−06 | 0.0003 | — |
| 13 | rs1572948 | 99039529 | 0.4025 | 1.94 | 0.0162 | 0.0004 | — |
| 13 | rs1018617 | 104839981 | 0.2486 | −0.2026 | 5.72E−05 | 2.93E−05 | — |
| 13 | rs656533 | 109591124 | 0.0903 | 0.6235 | 5.73E−06 | 0.0001 | — |
| 14 | rs10129606 | 21826110 | 0.3384 | 3.982 | 0.0003 | 0.0001 | BC063432 Z [symbol] |
| 14 | rs1952966 | 31508235 | 0.4611 | −0.2031 | 2.38E−06 | 0.0002 | — |
| 14 | rs4982155 | 33767943 | 0.2968 | 0.0928 | 0.2804 | 0.2036 | — |
| 14 | rs850246 | 56430538 | 0.3019 | 2.994 | 7.92E−08 | 2.61E−06 | −- |
| 14 | rs4444267 | 91917754 | 0.3217 | 0.1629 | 0.0003 | 3.93E−05 | SLC24A4 |
| 14 | rs8015515 | 100749638 | 0.4354 | 0.1141 | 0.0005 | 0.0003 | — |
| 14 | rs2250789 | 101485575 | 0.2946 | −0.1008 | 7.05E−06 | 1.61E−05 | — |
| 14 | rs10142224 | 105079690 | 0.0237 | 1.125 | 4.01E−06 | 0.0064 | — |
| 15 | rs1899922 | 33314940 | 0.2393 | 5.418 | 3.52E−05 | 2.88E−05 | — |
| 15 | rs1116123 | 51228469 | 0.2395 | 0.0967 | 6.55E−05 | 5.94E−07 | — |
| 15 | rs1715896 | 54615386 | 0.4752 | 1.548 | 0.0009 | 0.0003 | — |
| 15 | rs1002175 | 61473380 | 0.3211 | −1.487 | 0.007 | 0.0001 | — |
| 15 | rs768128 | 61482191 | 0.2517 | −1.184 | 0.0487 | 0.0027 | — |
| 15 | rs1501372 | 80255510 | 0.4102 | 0.1633 | 0.0002 | 0.0029 | EFTUD1 |
| 15 | rs2214076 | 89578780 | 0.263 | −3.217 | 0.0004 | 0.0022 | SV2B |
| 15 | rs9920002 | 92969194 | 0.1874 | 0.5335 | 7.51E−05 | 1.06E−05 | — |
| 15 | rs7174368 | 95724441 | 0.4133 | 0.3284 | 5.47E−05 | 3.05E−06 | — |
| 16 | rs4782058 | 16911739 | 0.2297 | 0.1447 | 8.79E−05 | 1.85E−05 | — |
| 16 | rs1453512 | 16925400 | 0.2302 | 0.1439 | 9.93E−05 | 2.57E−05 | — |
| 16 | rs8049145 | 17060587 | 0.3537 | −2.187 | 4.84E−05 | 0.0042 | — |
| 16 | rs8049145 | 17060587 | 0.3537 | −3.174 | 0.0002 | 0.0031 | — |
| 16 | rs7204375 | 60170344 | 0.1174 | 3.27 | 6.64E−05 | 5.85E−05 | — |
| 16 | rs10500535 | 64708404 | 0.1879 | 0.2157 | 4.85E−05 | 3.84E−06 | — |
| 16 | rs235092 | 64741598 | 0.3952 | 0.1229 | 0.0044 | 3.56E−05 | — |
| 16 | rs235141 | 64801256 | 0.2929 | 0.1495 | 0.0012 | 1.32E−05 | — |
| 16 | rs13332530 | 79656129 | 0.0017 | −2.364 | 4.94E−06 | 0.0507 | — |
| 16 | rs12597135 | 83663144 | 0.3592 | −0.1926 | 1.39E−05 | 0.0004 | KIAA0513 |
| 17 | rs2871275 | 6104186 | 0.4221 | 0.284 | 0.0022 | 0.0002 | — |
| 17 | rs6505476 | 30835146 | 0.3505 | 4.419 | 0.0002 | 0.0005 | — |
| 17 | rs1263982 | 47178422 | 0.4893 | 0.3318 | 0.0003 | 0.0012 | CA10 |
| 17 | rs16977009 | 67428119 | 0.3066 | 0.0867 | 7.88E−05 | 3.70E−05 | — |
| 17 | rs2158917 | 67437704 | 0.3002 | 0.0877 | 8.85E−05 | 4.44E−05 | — |
| 17 | rs4890055 | 76201820 | 0.1102 | −0.0708 | 0.5766 | 0.7705 | KIAA1303 B [symbol] |
| 18 | rs789071 | 998339 | 0.0858 | 0.5645 | 9.65E−05 | 5.65E−05 | — |
| 18 | rs789072 | 998417 | 0.0858 | 0.5645 | 9.65E−05 | 5.65E−05 | — |
| 18 | rs1720464 | 1003959 | 0.0858 | 0.5645 | 9.65E−05 | 5.65E−05 | — |
| 18 | rs789040 | 1007587 | 0.0863 | 0.6334 | 1.09E−05 | 6.92E−06 | — |
| 18 | rs7505855 | 29950073 | 0.4321 | 0.2955 | 0.0014 | 0.0001 | NOL4 |
| 18 | rs10503090 | 60161228 | 0.4304 | 2.681 | 0.0011 | 0.0094 | — |
| 18 | rs334428 | 67191559 | 0.3284 | −0.0999 | 0.0021 | 0.0002 | — |
| 18 | rs1614230 | 69999449 | 0.412 | 2.067 | 0.0073 | 0.0016 | — |
| 18 | rs869645 | 70003522 | 0.491 | −1.474 | 0.0033 | 0.006 | — |
| 19 | rs2335192 | 6284290 | 0.3019 | −0.1495 | 4.29E−06 | 3.22E−05 | ASAH3 |
| 19 | rs8109821 | 13143235 | 0.035 | 0.8169 | 4.91E−05 | 1.16E−05 | — |
| 19 | rs7249148 | 17560249 | 0.0832 | −0.1566 | 1.23E−05 | 1.17E−05 | — |
| 19 | rs1401926 | 21532772 | 0.4317 | 0.2235 | 1.94E−06 | 7.08E−06 | — |
| 19 | rs2061771 | 58602330 | 0.1343 | 5.565 | 5.78E−05 | 4.13E−05 | ZNF765 LO [symbol] |
| 20 | rs3916175 | 12928826 | 0.3708 | 2.473 | 0.0028 | 0.0007 | — |
| 20 | rs6033623 | 13060699 | 0.4526 | 0.3855 | 2.42E−05 | 8.37E−06 | SPTLC3 AK0 [symbol] |
| 20 | rs6078936 | 13066901 | 0.1337 | 0.5287 | 2.39E−05 | 7.48E−06 | SPTLC3 AK0 [symbol] |
| 20 | rs6078937 | 13078096 | 0.3426 | −0.4258 | 4.43E−06 | 1.73E−06 | SPTLC3 AK0 [symbol] |
| 20 | rs6041937 | 13099531 | 0.4706 | 0.356 | 6.27E−05 | 4.22E−05 | — |
| 20 | rs6109753 | 13138909 | 0.3753 | −2.129 | 5.52E−05 | 3.23E−06 | — |
| 20 | rs6105047 | 13140648 | 0.3702 | −2.156 | 4.59E−05 | 4.77E−06 | — |
| 20 | rs2269034 | 17397713 | 0.4441 | 0.0677 | 0.0009 | 0.0509 | BC040546 P |
| 20 | rs6082669 | 22248035 | 0.4977 | 8.731 | 0.0001 | 4.40E−05 | — |
| 20 | rs6071527 | 59176746 | 0.4673 | −0.3873 | 3.27E−05 | 2.22E−05 | — |
| 20 | rs944243 | 59605857 | 0.3713 | 2.625 | 0.0024 | 0.0021 | CDH4 AK09 [symbol] |
| 20 | rs11086147 | 61056624 | 0.4402 | 0.3155 | 0.0006 | 0.0004 | BC025312 C |
| 21 | rs9979091 | 33394730 | 0.2957 | 2.551 | 9.86E−06 | 6.61E−06 | — |
| 21 | rs3787771 | 37228011 | 0.39 | 0.5714 | 3.27E−05 | 1.17E−05 | HLCS |
| 21 | rs2835539 | 37230422 | 0.3962 | 0.5726 | 3.98E−05 | 5.75E−06 | HLCS |
| 21 | rs2837584 | 40662023 | 0.3792 | −3.317 | 5.95E−05 | 0.0062 | DSCAM |
| 22 | rs2096537 | 15474749 | 0.1476 | 2.817 | 7.12E−05 | 5.39E−05 | — |
| 22 | rs6519366 | 21239590 | 0.2223 | −3.716 | 5.73E−05 | 3.94E−05 | IGLC1 X578 [symbol] |
| 22 | rs2330191 | 21246106 | 0.2229 | −3.716 | 5.73E−05 | 3.94E−05 | IGLC1 X578 [symbol] |
| 22 | rs715594 | 21246960 | 0.2229 | −3.716 | 5.73E−05 | 3.94E−05 | IGLC1 X578 [symbol] |
| 22 | rs5759608 | 21832708 | 0.4927 | 0.0766 | 0.0001 | 0.0035 | RAB36 |
| 22 | rs5997024 | 24751591 | 0.1379 | 5.719 | 1.64E−06 | 0.0003 | MYO18B |
| 22 | rs16981226 | 24751793 | 0.1151 | 6.407 | 8.93E−07 | 0.0001 | MYO18B |
| 22 | rs2236005 | 24752980 | 0.1557 | 0.4698 | 6.30E−05 | 5.19E−05 | MYO18B |
| 22 | rs2236005 | 24752980 | 0.1557 | 5.114 | 9.26E−06 | 0.0003 | MYO18B |
| 22 | rs5997026 | 24761850 | 0.1422 | 0.2231 | 1.82E−06 | 3.30E−05 | — |
| 22 | rs6004913 | 24768495 | 0.1725 | 0.5896 | 3.94E−06 | 0.0018 | — |
| 22 | rs6004913 | 24768495 | 0.1725 | 0.2112 | 1.50E−06 | 0.0002 | — |
| 22 | rs6004913 | 24768495 | 0.1725 | 5.054 | 9.71E−06 | 0.0004 | — |
| 22 | rs131048 | 31311332 | 0.4165 | 0.2662 | 0.0053 | 0.0001 | SYN3 |
| 22 | rs139132 | 42913438 | 0.2223 | −0.0784 | 0.4391 | 0.6528 | PARVG |
| 22 | rs2032503 | 44567461 | 0.1896 | 0.0664 | 0.5226 | 0.7651 | ATXN10 |
| 23 | rs12688302 | 4201599 | 0.0045 | 0.8003 | 6.12E−08 | 0.0025 | — |
| 23 | rs196982 | 28618979 | 0.4347 | 0.1716 | 0.0012 | 0.0007 | — |
| 23 | rs12387837 | 28640215 | 0.4213 | 0.1891 | 0.0003 | 0.0014 | — |
| 23 | rs4829104 | 29483943 | 0.4785 | 0.5927 | 0.0002 | 0.0005 | IL1RAPL1 |
| 23 | rs2335993 | 35637531 | 0.4229 | 0.0573 | 0.0028 | 5.98E−05 | — |
| 23 | rs7055107 | 46456428 | 0.4373 | 1.618 | 0.0007 | 2.14E−05 | SLC9A7 |
| 23 | rs5922954 | 83434865 | 0.4734 | −0.1473 | 3.60E−06 | 0.0008 | — |
| 23 | rs1949393 | 86436628 | 0.3551 | 4.004 | 0.0008 | 0.0005 | — |
| 23 | rs5921062 | 97504619 | 0.2586 | 0.3697 | 0.0006 | 0.0098 | — |
| 23 | rs5911672 | 116370304 | 0.2917 | −0.1395 | 7.75E−05 | 6.56E−06 | — |
| 23 | rs5911732 | 116409123 | 0.2939 | −0.138 | 8.61E−05 | 9.10E−05 | — |
| 23 | rs5931073 | 136053015 | 0.3679 | 0.1706 | 0.0009 | 0.0019 | — |
| 23 | rs2873231 | 139742625 | 0.4818 | NA | NA | NA | — |
| 23 | rs180484 | 150068816 | 0.328 | −0.07 | 0.0005 | 0.0012 | — |
| 23 | rs6627710 | 151855148 | 0.3877 | −0.312 | 0.0018 | 0.0002 | ZNF185 BX5 |

| Chr | Nearby Genes(kb) | Trait |
|---|---|---|
| 1 | — | CHAQ_WK16 |
| 1 | SDC3(0) PUM1(54) U103(57) U103B(70) HBII-251(89) | CHAQ_WK16 |
| 1 | ST6GALNAC5(68) | AESINF |
| 1 | AK5(0) AK5(0) | Clearance |
| 1 | DENND2D(−86) CHI3L2(−44) CHIA(28) C1orf88(59) | CHAQ_WK16 |
| 1 | CHIA(−64) C1orf88(−33) OVGP1(29) WDR77(56) ATP5F1(64) | CSJC_WK16 |
| 1 | MTMR11(−94) OTUD7B(−19) VPS45(37) | CTJC_WK16 |
| 1 | OTUD7B(−43) VPS45(13) PLEKHO1(96) | CTJC_WK16 |
| 1 | VPS45(−26) PLEKHO1(−11) ANP32E(49) CA14(87) APH1A(94) | CDAS28_WK16 |
| 1 | VPS45(−26) PLEKHO1(−11) ANP32E(49) CA14(87) APH1A(94) | CTJC_WK16 |
| 1 | TPR(−67) C1orf27(−21) OCLM(−40) PDC(1) | CSJC_WK16 |
| 1 | TPR(−73) C1orf27(−26) OCLM(−46) PDC(0) | CSJC_WK16 |
| 1 | C1orf27(−57) OCLM(−77) PDC(−29) | CSJC_WK16 |
| 1 | — | CSJC_WK16 |
| 1 | PTGS2(95) | CSJC_WK16 |
| 1 | PTGS2(20) | CSJC_WK16 |
| 1 | PTGS2(19) | CSJC_WK16 |
| 1 | PTGS2(−2) | CSJC_WK16 |
| 1 | PTGS2(−4) | CSJC_WK16 |
| 1 | PTGS2(−19) | CSJC_WK16 |
| 1 | PTGS2(−33) | CSJC_WK16 |
| 1 | RRP15(−48) TGFB2(0) | CHAQ_WK16 |
| 1 | OR4F5(58) | CHAQ_WK16 |
| 1 | CHRM3(10) | CLDL_WK14 |
| 2 | — | CHAQ_WK16 |
| 2 | ALLC(−86) | CSJC_WK16 |
| 2 | ALLC(−87) | CDAS28_WK16 |
| 2 | ALLC(−87) | CSJC_WK16 |
| 2 | — | DAS28_B |
| 2 | — | DAS28_B |
| 2 | — | Clearance |
| 2 | — | CDAS28_WK16 |
| 2 | HPCAL1(97) | CHAQ_WK16 |
| 2 | HPCAL1(92) | CHAQ_WK16 |
| 2 | FAM110C(31) | Clearance |
| 2 | OSR1(−72) | ACR20_WK24 |
| 2 | — | CSJC_WK16 |
| 2 | — | CDAS28_WK16 |
| 2 | — | CDAS28_WK16 |
| 2 | REG1B(−95) REG1A(−60) REG3A(−23) | CCRP_WK08 |
| 2 | KCMF1(−31) TCF7L1(43) | CCRP_WK08 |
| 2 | — | AESINF |
| 2 | MBD5(56) | CLDL_WK14 |
| 2 | SCN7A(0) | CTJC_WK16 |
| 2 | SCN7A(0) | CTJC_WK16 |
| 2 | — | CLDL_WK14 |
| 2 | — | CLDL_WK14 |
| 2 | ZNF533(0) | CHAQ_WK16 |
| 2 | CALCRL(−35) TFPI(45) TFPI(57) | DAS28_B |
| 2 | — | CLDL_WK14 |
| 2 | — | DAS28_B |
| 2 | ERBB4(0) | DAS28_B |
| 2 | TRAF3IP1(0) ASB1(29) | Clearance |
| 2 | TRAF3IP1(−41) ASB1(0) | Clearance |
| 3 | CNTN6(0) | CCRP_WK08 |
| 3 | — | CCRP_WK08 |
| 3 | CNTN4(20) | Clearance |
| 3 | CNTN4(0) IL5RA(78) IL5RA(98) | ACR20_WK24 |
| 3 | CNTN4(−2) IL5RA(11) IL5RA(31) TRNT1(69) CRBN(91) | DAS28_B |
| 3 | — | CHAQ_WK16 |
| 3 | ZNF659(0) | CCRP_WK08 |
| 3 | RARB(73) | CSJC_WK16 |
| 3 | LRRC3B(−41) | CLDL_WK14 |
| 3 | LRRC3B(−99) | CLDL_WK14 |
| 3 | — | CTJC_WK16 |
| 3 | CTNNB1(−3) | CSJC_WK16 |
| 3 | CTNNB1(−3) | CTJC_WK16 |
| 3 | TMEM16K(0) | CHAQ_WK16 |
| 3 | ARHGEF3(−88) | CCRP_WK08 |
| 3 | ALCAM(−62) CBLB(24) | DAS28_B |
| 3 | — | CSJC_WK16 |
| 3 | CD86(−97) CASR(37) | CTJC_WK16 |
| 3 | C3orf36(−70) SLCO2A1(0) | AESINF |
| 3 | — | CTJC_WK16 |
| 3 | — | CDAS28_WK16 |
| 3 | NAALADL2(0) | CDAS28_WK16 |
| 3 | — | DAS28_B |
| 3 | ST6GAL1(82) | ACR20_WK24 |
| 3 | TP73L(50) | Clearance |
| 4 | — | Clearance |
| 4 | CHIC2(−29) GSH2(6) | CCRP_WK08 |
| 4 | HNRPD(−10) HNRPDL(41) ENOPH1(46) | CTJC_WK16 |
| 4 | HSD17B13(0) HSD17B11(29) | CHAQ_WK16 |
| 4 | — | ACR20_WK24 |
| 4 | — | CCRP_WK08 |
| 5 | — | CDAS28_WK16 |
| 5 | — | CDAS28_WK16 |
| 5 | — | CDAS28_WK16 |
| 5 | — | DAS28_B |
| 5 | — | DAS28_B |
| 5 | — | DAS28_B |
| 5 | — | Clearance |
| 5 | RXFP3(−60) SLC45A2(−14) AMACR(0) AMACR(0) C1QTNF3(21) | CCRP_WK08 |
| 5 | — | CSJC_WK16 |
| 5 | COX7C(3) | CHAQ_WK16 |
| 5 | PCSK1(−48) | Clearance |
| 5 | — | CLDL_WK14 |
| 5 | — | CLDL_WK14 |
| 5 | — | CCRP_WK08 |
| 5 | KLHL3(0) hsa-mir-874(−17) HNRPA0(87) | CCRP_WK08 |
| 5 | GALNT10(−50) SAP30L(−11) HAND1(8) | Clearance |
| 5 | — | ACR20_WK24 |
| 5 | — | CHAQ_WK16 |
| 6 | FAM50B(−62) | CHAQ_WK16 |
| 6 | FAM50B(−62) | CTJC_WK16 |
| 6 | — | AESINF |
| 6 | LRFN2(0) | CSJC_WK16 |
| 6 | TREML1(−49) TREM2(−40) TREML2(−2) TREML4(24) TREM1(72) | AESINF |
| 6 | BAI3(0) | ACR20_WK24 |
| 6 | — | CDAS28_WK16 |
| 6 | — | ACR20_WK24 |
| 6 | TBX18(11) | CSJC_WK16 |
| 6 | SIM1(36) | AESINF |
| 6 | ESR1(−66) SYNE1(0) SYNE1(0) SYNE1(0) | DAS28_B |
| 6 | MAP3K4(0) | CCRP_WK08 |
| 6 | MAP3K4(0) AGPAT4(82) | CCRP_WK08 |
| 6 | MAP3K4(0) AGPAT4(61) | CCRP_WK08 |
| 6 | PDE10A(0) | CDAS28_WK16 |
| 7 | ICA1(−34) | CTJC_WK16 |
| 7 | — | DAS28_B |
| 7 | SNX13(85) | CLDL_WK14 |
| 7 | — | CLDL_WK14 |
| 7 | ABCB5(−43) SP8(−13) | CHAQ_WK16 |
| 7 | NEUROD6(46) | CCRP_WK08 |
| 7 | — | CCRP_WK08 |
| 7 | — | CCRP_WK08 |
| 7 | GTF2IRD1(0) | DAS28_B |
| 7 | GTF2IRD1(0) | DAS28_B |
| 7 | CACNA2D1(0) | CCRP_WK08 |
| 7 | — | CDAS28_WK16 |
| 7 | AKAP9(−85) CYP51A1(−61) KRIT1(4) | CDAS28_WK16 |
| 7 | FLJ42280(36) | CLDL_WK14 |
| 7 | RINT1(−83) | DAS28_B |
| 7 | NRF1(51) | CCRP_WK08 |
| 7 | HSPC268(48) LUC7L2(67) | CCRP_WK08 |
| 7 | CNTNAP2(0) | CSJC_WK16 |
| 8 | — | CHAQ_WK16 |
| 8 | BLK(−75) GATA4(69) | DAS28_B |
| 8 | DPYSL2(−55) ADRA1A(37) ADRA1A(45) ADRA1A(54) | Clearance |
| | ADRA1A(59) | |
| 8 | DPYSL2(−60) ADRA1A(32) ADRA1A(40) ADRA1A(50) | Clearance |
| | ADRA1A(54) | |
| 8 | — | Clearance |
| 8 | EYA1(0) | CTJC_WK16 |
| 8 | CNBD1(11) | CLDL_WK14 |
| 8 | PGCP(−23) | CTJC_WK16 |
| 8 | — | CDAS28_WK16 |
| 8 | NSMCE2(−82)TRIB1(−13) | CTJC_WK16 |
| 8 | — | CTJC_WK16 |
| 8 | — | CSJC_WK16 |
| 9 | — | CHAQ_WK16 |
| 9 | FLJ00038(4) | CCRP_WK08 |
| 9 | — | CTJC_WK16 |
| 9 | C9orf138(0) RRAGA(97) | CLDL_WK14 |
| 9 | — | AESINF |
| 9 | — | CHAQ_WK16 |
| 9 | PCSK5(0) | CSJC_WK16 |
| 9 | ROR2(0) | AESINF |
| 9 | PRG-3(5) | Clearance |
| 9 | FREQ(−25) | CCRP_WK08 |
| 9 | FREQ(−30) | CCRP_WK08 |
| 10 | ADARB2(−91) | AESINF |
| 10 | PTER(−29) C1QL3(−20) RSU1(52) RSU1(52) | ACR20_WK24 |
| 10 | — | CCRP_WK08 |
| 10 | PRKG1(0) | CSJC_WK16 |
| 10 | PRKG1(0) | CSJC_WK16 |
| 10 | TUBB8(82) | Clearance |
| 10 | PCDH15(0) | Clearance |
| 10 | TUBB8(82) | CCRP_WK08 |
| 10 | CTNNA3(0) | CCRP_WK08 |
| 10 | SGPL1(−98) PCBD1(−87) | Clearance |
| 10 | ZNF503(32) | CDAS28_WK16 |
| 10 | TUBB8(82) | CDAS28_WK16 |
| 10 | KCNMA1(−14) | CSJC_WK16 |
| 10 | KCNMA1(−16) | CSJC_WK16 |
| 10 | KCNMA1(−23) | CSJC_WK16 |
| 10 | NRG3(0) | CDAS28_WK16 |
| 10 | — | CHAQ_WK16 |
| 10 | — | CHAQ_WK16 |
| 10 | DOCK1(0) | ACR20_WK24 |
| 10 | PPP2R2D(0) BNIP3(23) | ACR20_WK24 |
| 11 | EIF4G2(−75) U97(−80) ZBED5(−27) | CSJC_WK16 |
| 11 | GALNTL4(0) | CTJC_WK16 |
| 11 | USP47(−39) DKK3(0) | CHAQ_WK16 |
| 11 | USP47(−39) DKK3(0) | CTJC_WK16 |
| 11 | USP47(−42) DKK3(0) | CHAQ_WK16 |
| 11 | USP47(−60) DKK3(−11) | CHAQ_WK16 |
| 11 | HTATIP(−92) RNASEH2C(−90) DKFZp761E198(−30) OVOL1(−15) | CHAQ_WK16 |
| | FLJ3 [symbol] | |
| 11 | LRRC32(5) | CSJC_WK16 |
| 11 | CNTN5(0) | CSJC_WK16 |
| 11 | CNTN5(0) | CLDL_WK14 |
| 11 | — | CHAQ_WK16 |
| 11 | — | Clearance |
| 11 | — | Clearance |
| 11 | ZBTB16(0) HAR40(5) NNMT(73) | CDAS28_WK16 |
| 11 | TRIM29(97) | DAS28_B |
| 11 | OPCML(−73) OPCML(0) | CSJC_WK16 |
| 11 | NCAPD3(−28) VPS26B(−6) THYN1(0) THYN1(0) ACAD8(1) | CHAQ_WK16 |
| 12 | TSPAN9(−46) | CLDL_WK14 |
| 12 | GALNT8(−30) KCNA6(7) | CCRP_WK08 |
| 12 | KLRB1(−62) CLEC2D(0) CLEC2D(0) DCAL1(52) CD69(83) | ACR20_WK24 |
| 12 | KLRB1(−74) CLEC2D(0) CLEC2D(0) DCAL1(40) CD69(71) | ACR20_WK24 |
| 12 | FLJ32894(79) | Clearance |
| 12 | AVPR1A(−82) | CCRP_WK08 |
| 12 | CART1(−60) | ACR20_WK24 |
| 12 | — | CTJC_WK16 |
| 12 | CLLU1OS(15) CLLU1(18) | CLDL_WK14 |
| 12 | CLLU1OS(0) CLLU1(0) | CLDL_WK14 |
| 12 | RBM19(28) | CCRP_WK08 |
| 12 | — | CLDL_WK14 |
| 13 | KIAA0774(0) | CCRP_WK08 |
| 13 | — | CHAQ_WK16 |
| 13 | — | Clearance |
| 13 | — | CLDL_WK14 |
| 13 | — | CDAS28_WK16 |
| 13 | — | AESINF |
| 13 | KLF12(0) KLF12(0) | AESINF |
| 13 | — | CSJC_WK16 |
| 13 | — | DAS28_B |
| 13 | — | CCRP_WK08 |
| 13 | — | CCRP_WK08 |
| 13 | — | CCRP_WK08 |
| 13 | MBNL2(91) MBNL2(91) | CCRP_WK08 |
| 13 | MBNL2(87) MBNL2(87) | CCRP_WK08 |
| 13 | TM9SF2(−26) CLYBL(17) | CTJC_WK16 |
| 13 | — | DAS28_B |
| 13 | COL4A1(9) | CCRP_WK08 |
| 14 | — | AESINF |
| 14 | — | DAS28_B |
| 14 | — | CCRP_WK08 |
| 14 | OTX2(−88) | CSJC_WK16 |
| 14 | SLC24A4(0) | DAS28_B |
| 14 | — | AESINF |
| 14 | PPP2R5C(−24) DYNC1H1(15) | Clearance |
| 14 | MTA1(−72) CRIP2(−62) CRIP1(−53) C14orf80(−43) TMEM121(−12) | CCRP_WK08 |
| 15 | — | AESINF |
| 15 | — | Clearance |
| 15 | — | ACR20_WK24 |
| 15 | — | CSJC_WK16 |
| 15 | — | CSJC_WK16 |
| 15 | EFTUD1(0) DKFZp666G057(86) | DAS28_B |
| 15 | SV2B(0) | CTJC_WK16 |
| 15 | — | ACR20_WK24 |
| 15 | — | CCRP_WK08 |
| 16 | — | CHAQ_WK16 |
| 16 | — | CHAQ_WK16 |
| 16 | XYLT1(49) | CSJC_WK16 |
| 16 | XYLT1(49) | CTJC_WK16 |
| 16 | CDH8(74) | CSJC_WK16 |
| 16 | — | DAS28_B |
| 16 | POLR3K(37) C16orf33(43) RHBDF1(48) MPG(68) MPG(69) | DAS28_B |
| 16 | — | DAS28_B |
| 16 | C16orf61(−67) CENPN(−42) ASCIZ(−20) C16orf46(−1) GCSH(17) | DAS28_B |
| 16 | ZDHHC7(−81) KIAA0513(0) FAM92B(27) | DAS28_B |
| 17 | — | CDAS28_WK16 |
| 17 | SLFN12(−60) SLFN13(−38) mgU6-47(89) PEX12(91) | AESINF |
| 17 | CA10(0) | CDAS28_WK16 |
| 17 | — | Clearance |
| 17 | — | Clearance |
| 17 | KIAA1303(0) | CCRP_WK08 |
| 18 | ADCYAP1(−98) | CCRP_WK08 |
| 18 | — | CCRP_WK08 |
| 18 | — | CCRP_WK08 |
| 18 | — | CCRP_WK08 |
| 18 | NOL4(0) | CDAS28_WK16 |
| 18 | — | CTJC_WK16 |
| 18 | — | CHAQ_WK16 |
| 18 | FBXO15(−45) C18orf55(−22) CYB5A(72) CYB5A(75) | CTJC_WK16 |
| 18 | FBXO15(−49) C18orf55(−26) CYB5A(68) CYB5A(71) | CSJC_WK16 |
| 19 | MLLT1(−53) ASAH3(0) CLPP(28) ALKBH7(39) PSPN(42) | CHAQ_WK16 |
| 19 | NFIX(−72) LYL1(−70) TRMT1(−55) BTBD14B(−33) STX10(−21) | CCRP_WK08 |
| 19 | SLC27A1(−83) PGLS(−67) FAM129C(−34) GLT25D1(−6) | Clearance |
| | UNC13A(12) | |
| 19 | ZNF429(−19) | CLDL_WK14 |
| 19 | ZNF765(0) ZNF761(47) ZNF813(60) | AESINF |
| 20 | C20orf38(9) | CTJC_WK16 |
| 20 | C20orf38(−82) | CDAS28_WK16 |
| 20 | C20orf38(−88) | CDAS28_WK16 |
| 20 | — | CDAS28_WK16 |
| 20 | — | CDAS28_WK16 |
| 20 | — | CSJC_WK16 |
| 20 | — | CSJC_WK16 |
| 20 | PCSK2(0) BFSP1(25) | Clearance |
| 20 | — | AESINF |
| 20 | CDH4(84) | CDAS28_WK16 |
| 20 | CDH4(0) | CTJC_WK16 |
| 20 | TCFL5(−93) DIDO1(−43) DIDO1(−43) C20orf11(−9) C20orf59(0) | CDAS28_WK16 |
| 21 | OLIG2(−72) OLIG1(−28) | CSJC_WK16 |
| 21 | HLCS(0) DSCR6(72) | ACR20_WK24 |
| 21 | HLCS(0) DSCR6(70) | ACR20_WK24 |
| 21 | DSCAM(0) | CTJC_WK16 |
| 22 | CESK1(−21) | CSJC_WK16 |
| 22 | SUHW2(−65) SUHW1(−39) PRAME(−10) GGTL4(79) | CTJC_WK16 |
| 22 | SUHW2(−72) SUHW1(−46) PRAME(−16) GGTL4(72) | CTJC_WK16 |
| 22 | — | CTJC_WK16 |
| 22 | RTDR1(−20) GNAZ(−37) RAB36(0) BCR(20) | Clearance |
| 22 | MYO18B(0) | CTJC_WK16 |
| 22 | MYO18B(0) | CTJC_WK16 |
| 22 | MYO18B(0) | ACR20_WK24 |
| 22 | MYO18B(0) | CTJC_WK16 |
| 22 | MYO18B(−4) | CHAQ_WK16 |
| 22 | MYO18B(−11) | CDAS28_WK16 |
| 22 | MYO18B(−11) | CHAQ_WK16 |
| 22 | MYO1SB(−11) | CTJC_WK16 |
| 22 | — | CDAS28_WK16 |
| 22 | — | CCRP_WK08 |
| 22 | ATXN10(0) | CCRP_WK08 |
| 23 | — | Clearance |
| 23 | IL1RAPL1(98) | CLDL_WK14 |
| 23 | IL1RAPL1(77) | CLDL_WK14 |
| 23 | ILIRAPL1(0) | ACR20_WK24 |
| 23 | — | Clearance |
| 23 | SLC9A7(0) | ACR20_WK24 |
| 23 | CXorf43(28) | CHAQ_WK16 |
| 23 | — | AESINF |
| 23 | — | CDAS28_WK16 |
| 23 | — | CHAQ_WK16 |
| 23 | — | CHAQ_WK16 |
| 23 | — | CLDL_WK14 |
| 23 | CDR1(−48) | DAS28_B |
| 23 | GPR50(26) | Clearance |
| 23 | NSDHL(−66) PNMA5(54) | CDAS28_WK16 |

[symbol]

indicates data missing or illegible when filed

What is claimed is:

1. A method of identifying a rheumatoid arthritis patient that is a candidate for treatment with an human interleukin-6 receptor antibody or patient that should be excluded from treatment, the method comprising:
providing a nucleic acid sample from the patient;
analyzing the nucleic acid sample for the presence of at least one single nucleotide polymorphism (SNP) biomarker set forth in Table 1 that is associated with a therapeutic response to treatment with IL-6 receptor antibody, or a polymorphism in strong linkage disequilibrium with a SNP biomarker set forth in Table 1, wherein the presence of the associated biomarker is indicative of a patient that is a candidate for treatment with the human interleukin-6 receptor antibody; or a patient that should be excluded from treatment.

2. The method of claim 1, wherein the at least one biomarker is set forth in Table 2.

3. The method of claim 2, wherein the SNP biomarker is rs6004913 or a polymorphism in strong linkage disequilibrium with rs6004913, or the SNP biomarker is rs6078937 or a polymorphism in strong linkage disequilibrium with rs6078937.

4. The method of claim 2, wherein the biomarkers comprise the seven biomarkers set forth in Table 2.

5. The method of claim 1, wherein the nucleic acid sample is analyzed for the presence of at least two biomarkers set forth in Table 1 that are associated with therapeutic response to treatment with IL-6 receptor antibody.

6. The method of claim 5, wherein the at least two biomarkers are rs6004913 or a polymorphism in strong linkage disequilibrium with rs6004913, and rs6078937 or a polymorphism in strong linkage disequilibrium with rs6078937.

7. The method of claim 1, wherein the nucleic acid sample is analyzed for the presence of at least 10 biomarkers associated with therapeutic response to treatment with IL-6 receptor antibody.

8. The method of claim 1, wherein the nucleic acid sample is analyzed for the presence of at least 50 biomarkers associated with therapeutic response to treatment with IL-6 receptor antibody.

9. The method of claim 1, wherein the nucleic acid sample comprises genomic DNA.

10. The method of claim 1, wherein the step of analyzing the nucleic acid sample comprises sequencing the nucleic acid.

11. The method of claim 1, wherein the step of analyzing the nucleic acid sample comprises determining the presence of the SNP allele using an allele-selective oligonucleotide probe.

12. The method of claim 1, further comprising recording the correlation of the presence of the SNP with a positive response to treatment with IL-6 receptor antibody.

13. The method of claim 1, further comprising administering IL-6 receptor antibody to the patient.

14. A diagnostic device comprising probes attached to a solid surface to detect two or more SNPs set forth in Table 1.

15. The diagnostic device of claim 14, wherein the two are more SNPs are set forth in Table 2.

16. A kit comprising allele-specific oligonucleotides to detect a major allele and a minor allele for two or more SNPs set forth in Table 1.

17. The kit of claim 16, wherein the two or more SNPs are set forth in Table 2.
